(19) United States
(12) Patent Application Publication
(10) Pub. No.: US 2025/0288524 A1
(43) Pub. Date: Sep. 18, 2025

(54) SPRAYED MULTI ADSORBED-DROPLET REPOSING TECHNOLOGY (SMART)

(71) Applicant: Board of Regents, The University of Texas System, Austin, TX (US)

(72) Inventors: Mohammed MANIRUZZAMAN, Austin, TX (US); Jiawei WANG, Austin, TX (US); Niloofar Heshmati AGHDA, Austin, TX (US); Yu ZHANG, Austin, TX (US)

(73) Assignee: Board of Regents, The University of Texas System, Austin, TX (US)

(21) Appl. No.: 18/576,848

(22) PCT Filed: Jul. 7, 2022
(86) PCT No.: PCT/US2022/036336

§ 371 Date: Jan. 5, 2024

Related U.S. Application Data
(60) Provisional application No. 63/219,258, filed on Jul. 7, 2021.

(51) Int. Cl. A61K 9/16; A61K 9/107; A61K 9/48; A61K 31/198; A61K 31/7088; A61K 35/12; A61K 35/74; A61K 36/06; A61K 38/16

(52) U.S. Cl. CPC A61K 9/1647; A61K 9/107; A61K 9/1682; A61K 9/48; A61K 31/198; A61K 31/7088; A61K 35/12; A61K 35/74; A61K 36/06; A61K 38/16

(57) ABSTRACT

Described are techniques, systems, and methods include those employing pneumatic, pressure assisted, extrusion-based 3D printing and emulsion evaporation, emulsion diffusion, nanoprecipitation, desolvation, gelation, spray-based atomization, etc. for fabricating loaded microparticles or nanoparticles that encapsulate an active pharmaceutical ingredient or live cells into a biocompatible polymer or pharmaceutical excipients. The techniques provide for encapsulation of a variety of substances including proteins, plasmid DNA, lipophilic pharmaceutical compositions, hydrophilic pharmaceutical compositions, live cells, and/or cellular components into polymeric microparticles or nanoparticles. The particles loaded with active pharmaceutical ingredients can be used for the treatment of different diseases or conditions. The particles loaded with live cells can be used for disease treatment, but can also be used for securely storing the live cells in a stable condition for transport and later use in inoculating fermentation systems, for example, to generate recombinant proteins.

### CROSS-REFERENCE TO RELATED APPLICATIONS

This application claims the benefit of and priority to U.S. Provisional Application No. 63/219,258, filed on Jul. 7, 2021, which is hereby incorporated by reference in its entirety.

### FIELD

This invention is in the field of microparticles and nanoparticles and delivery of live cells, biologics, or active pharmaceutical ingredients by microparticles and nanoparticles. This invention relates generally to an extrusion-based printing process and emulsion evaporation method as well as any other suitable methods such as emulsion-evaporation/diffusion, nanoprecipitation, desolvation, gelation, spray-based atomization, etc., for formulating polymeric microparticles or nanoparticles, microparticle and nanoparticle formulations, and associated systems.

### BACKGROUND

In the past few decades, polymeric biodegradable microparticles (MPs) have attracted worldwide research interests, and have emerged as a powerful tool for the delivery of pharmaceutical reagents. The effectiveness and function of biodegradable MPs depend on several physiochemical properties, including size, surface charge, shape, as well as hydrophobicity, and hydrophilicity. Formulated MPs allow the encapsulation of a variety of agents, including proteins, plasmid DNA, lipophilic and hydrophilic drugs. Moreover, fabricated MPs are suitable for many administration routes, such as inhalation, injection, and oral delivery. Different ligands and antibodies can also be attached to the MP surface for targeted drug delivery. In addition, PEGylated MPs with stealth properties are developed to increase the circulation time in vivo for improved therapeutic efficiency.

Conventional microparticle formulation techniques involve solvent displacement and emulsion evaporation techniques. The basis of solvent displacement involves an organic phase (solvent mix) being added into the aqueous phase. The solvent phase tends to have a diffusion effect, whereas the polymer automatically tends to collapse forming nanoparticles or microparticles that can encapsulate active ingredients contained in the organic phase. On the other hand, the solvent emulsion evaporation method is mainly a multi-step process: the emulsification of a polymer solution containing the encapsulated substance, followed by particle hardening through solvent evaporation and polymer precipitation. During emulsification, the polymer solution is broken into micro-sized emulsion droplets by the shear stress generated either by a sonicator or a homogenizer in the presence of a surfactant. Furthermore, an ice bath is needed to cool down the sonicated emulsion due to the enormous heat generated during this process.

### SUMMARY

Biodegradable microparticles have been extensively investigated over the past few decades, which have emerged as a powerful tool for the delivery of pharmaceutical reagents. Conventional microparticle formulation techniques involve solvent displacement and emulsion evaporation techniques. This disclosure provides methods, systems, compositions, and techniques referred to herein as sprayed multi adsorbed-droplet reposing technology (SMART) that combines extrusion-based printing and emulsion evaporation techniques to fabricate novel polymeric particles, such as microparticles and nanoparticles, such as comprising polymeric poly(lactide-co-glycolide) (PLGA). PLGA is a biocompatible and biodegradable FDA-approved copolymer, which can be hydrolyzed into lactic and glycolic acid monomers.

Polymeric nanoparticles in the size range of 10-200 nm may be suitable for biomedical applications that require local or systemic administration, interaction with diseased tissues at the cellular and molecular level, and uptake into cells. The high surface area and chemical versatility of these nanoparticles may enable surface functionalization, with targeting ligands that can enhance transport across physiological barriers and provide specificity toward molecular targets characteristic of diseased tissues. At the same time, because of their macromolecular size, nanoparticles can readily act as carriers for controlled delivery of therapeutic agents, contrast agents or other cargo. As such, nanoparticles are expected to be useful for future diagnostic, therapeutic and theranostic technologies. In the field of nanomedicine, polymeric nanoparticles have mostly been used for drug delivery approaches to facilitate the pulmonary, oral, transdermal and intravenous delivery of therapeutic agent for the treatment of cancer, infection diseases, and inflammation diseases. While to date, only a few nanoparticle-based systems have entered the market as therapeutics or biotechnological tools, it is expected that nanotechnology may feature prominently in health care in the near future. Several nanoparticle-based formulations have been approved by the FDA, including liposomal doxorubicin (Doxil), liposomal daunorubicin (DaunoXome), liposomal amphotericin B (Abelcet, Amphotec, and AmBisome), and paclitaxel-loaded albumin nanoparticles (Abraxane). Despite the high versatility and long history of polymeric materials in medicine, to date, no therapeutic nano-scaled particle formulations based on biodegradable polyesters such as poly(lactic-co-glycolic acid) (PLGA) have been approved by the FDA for clinical use. As diagnostic agents, polymeric nanoparticles can be used as contrast agents for biomedical imaging, labeling probes for biomarker or pathogen detection, or as capture agents for the separation of biological molecules or cells. Conjugates of polymeric nanoparticles with antibodies, aptamers and oligonucleotides enable the detection of the disease biomarkers. Nanoparticles can also be incorporated into biomedical device coatings or blended as nanocomposites for the preparation of drug eluting stents, tissue engineered scaffolds, or antibacterial coatings that require the controlled release of active agents, high porosity, or nano-scaled topologies. Polymeric nanoparticles have also been used for separation and purification in bioprocesses. Stimuli-responsive nanoparticles have also been used as nanofillers to provide tunable porosity within gels for the separation of biological molecules through electrophoresis. Polymeric nanoparticles have also been utilized in the cosmetic industry for the delivery of skin care, antiacne and antioxidant agents to the pores of the skin. Highly permeable hair products based on polymer nanoparticles are being fabricated to deliver blood flow acceleration, cell activation and androgen suppression agents. The food industry can also benefit from polymer-based nanoparticles for the encapsulation of phytonutrients and prebiotics. The use of polymeric nanoparticles has been reported in environmental applications, for instance, in the bioremediation of soil.

Compared to conventional emulsion evaporation methods, the techniques described herein are suitable for encapsulating heat-sensitive drugs, biomolecules, and live microorganisms such as bacteria, yeasts, etc. For example, by using the shear force exerted by a syringe nozzle rather than sonication energy, micro- and/or nano-sized emulsion droplets can be created, eliminating the emulsion cooling step. Furthermore, the shear force provided can be consistent and controllable, as its intensity may depend on the syringe nozzle size, printing speed, and printing pressure, which can be carefully controlled during the extrusion-based printing process.

Another advantage of the methods, systems, compositions, and techniques described herein is the ability to incorporate live cells and bacteria during the particle formulation process. Aspects described herein can combine bioprinting with particulate-based drug delivery systems in a ‘one-step’ process, useful for a variety of applications and drug delivery for different disease treatments, including particulate-based drug delivery in stem cell therapy.

In an aspect, methods are described herein, such as methods for preparing particles (e.g., microparticles and/or nanoparticles). In some examples, a method of this aspect comprises preparing an emulsion comprising water, a polymer or a non-polymeric excipient, a solvent, and an active pharmaceutical ingredient; printing the emulsion using an extrusion-based printing method to generate a plurality of droplets including particles having diameters of from 10 nm to 1000 μm and comprising the polymer or the non-polymeric excipient and the active pharmaceutical ingredient; and collecting the plurality of droplets. In some examples, the extrusion-based printing method subjects the emulsion to shear forces that separate the emulsion into the plurality of droplets including particles. Optionally the particles prepared according to this aspect may be subjected to further processing. For example, methods of this aspect may further comprise subjecting the droplets to evaporation conditions to evaporate from the droplets and leave the particles. Methods of this aspect may further comprise washing the plurality of particles, for example. Methods of this aspect may further comprise lyophilizing the plurality of droplets or the particles. In some examples, the particles may have diameters of from about 10 nm to about 1 mm or larger. For example, the particles may have diameters of from 10 nm to 20 nm, from 20 nm to 30 nm, from 30 nm to 40 nm, from 40 nm to 50 nm, from 50 nm to 60 nm, from 60 nm to 70 nm, from 70 nm to 80 nm, from 80 nm to 90 nm, from 90 nm to 100 nm, from 100 nm to 150 nm, from 150 nm to 200 nm, from 200 nm to 250 nm, from 250 nm to 300 nm, from 300 nm to 350 nm, from 350 nm to 400 nm, from 400 nm to 450 nm, from 450 nm to 500 nm, from 500 nm to 600 nm, from 600 nm to 700 nm, from 700 nm to 800 nm, from 800 nm to 900 nm, from 900 nm to 1 μm, from 1 μm to 2 μm, from 2 μm to 3 μm, from 3 μm to 4 μm, from 4 μm to 5 μm, from 5 μm to 6 μm, from 6 μm to 7 μm, from 7 μm to 8 μm, from 8 μm to 9 μm, from 9 μm to 10 μm, from 10 μm to 20 μm, from 20 μm to 30 μm, from 30 μm to 40 μm, from 40 μm to 50 μm, from 50 μm to 60 μm, from 60 μm to 70 μm, from 70 μm to 80 μm, from 80 μm to 90 μm, from 90 μm to 100 μm, from 100 μm to 150 μm, from 150 μm to 200 μm, from 200 μm to 250 μm, from 250 μm to 300 μm, from 300 μm to 350 μm, from 350 μm to 400 μm, from 400 μm to 450 μm, from 450 μm to 500 μm, from 500 μm to 600 μm, from 600 μm to 700 μm, from 700 μm to 800 μm, from 800 μm to 900 μm, or from 900 μm to 1 mm.

A variety of different emulsions may be used for preparing particles (e.g., microparticles and/or nanoparticles) according to the aspects described herein. For example, the emulsion may comprise a water-in-oil emulsion, an oil-in-water emulsion, or a water-in-oil-in-water emulsion. Preparing the emulsion may comprise preparing a water-in-oil emulsion, an oil-in-water emulsion, or a water-in-oil-in-water emulsion. In a specific example, preparing the emulsion comprises preparing a primary emulsion comprising a water-in-oil emulsion or an oil-in-water emulsion, and preparing a secondary emulsion comprising a water-in-oil-in-water emulsion. Other components may be included in the emulsion. For example, the emulsion may comprise or further comprise one or more of a cosolvent, a surfactant, a preservative, live cells, cellular components, an additional active ingredient, a salt, a preservative, a protein, a peptide, an amino acid, or a nucleic acid component.

The methods described herein may generally be useful for preparing particles (e.g., microparticles and/or nanoparticles) containing any desirable active ingredient. Without limitation, example active ingredients may comprise a protein, an antibody, a nucleic acid, messenger ribonucleic acid (mRNA) molecules, a lipid nanoparticle, clustered regularly interspaced short palindromic repeats (CRISPR)-associated protein 9 (Cas9), transcription activator-like effector nucleases (TALENs), zinc-finger nucleases (ZFNs), homing endonucleases or meganucleases, a growth factor, a plasmid, a hydrophilic pharmaceutical, a lipophilic pharmaceutical, a viral particle, a virus-like particle, a live yeast cell, a live recombinant yeast cell, a live fungus, a live bacterial cell, a live recombinant bacterial cell, a live insect cell, a live mammalian cell, or a live mesenchymal stem cell. In some examples, a weight ratio of the active pharmaceutical ingredient to the polymer or the non-polymeric excipient in the emulsion is from 1:8 to 1:15, such as from 1:8 to 1:9, from 1:9 to 1:10, from 1:10 to 1:11, from 1:11 to 1:12, from 1:12 to 1:13, from 1:13 to 1:14, or from 1:14 to 1:15.

For particles comprising a polymer, the polymer may be a biocompatible polymer, a biodegradable polymer, or any pharmaceutically acceptable polymer. Example biodegradable polymers include, but are not limited to, poly(lactide-co-glycolide), polylactide (PLA), polyglycolide (PGA), polycaprolactone (PCL), pluronic F127, sodium alginate, hyaluronic acid, chitosan, cyclodextrin, dextran, agarose, gelatin, albumin, collagen, lipids, a polyethylene glycol (PEG) derivative, a pharmaceutical grade polymer, poly(hydroxy butyrate), poly(β-malic acid), or poly(L-lysine).

For particles comprising a non-polymeric excipient, the non-polymeric excipient may be a hydrophilic substance or a hydrophobic substance. Example non-polymeric excipients include, but are not limited to, a non-reducing sugar, such as trehalose, or sucrose, a polyol, such as mannitol, sorbitol, xylitol, or an amino acid, such as leucine, or L-arginine.

The particles (e.g., microparticles and/or nanoparticles) can be prepared using any suitable printing parameters and any suitable environmental parameters. In some examples, the printing may occur at ambient conditions (e.g., at atmospheric pressure and at room temperature). Temperatures for collecting the plurality of droplets may correspond to ambient temperature or cryogenic temperatures. For example, collecting the plurality of droplets comprises receiving the plurality of droplets on a surface having a temperature of from about −200° C. to about −78° C. or at room temperature or from about 4° C. to about 50° C. In some examples, the extrusion-based printing method subjects the emulsion to a pressure of from 10 kPa to 700 kPa, such as from 10 kPa to 600 kPa, from 10 kPa to 500 kPa, from 10 kPa to 400 kPa, from 10 kPa to 300 kPa, from 10 kPa to 200 kPa, from 10 kPa to 100 kPa, from 10 kPa to 20 kPa, from 20 kPa to 30 kPa, from 30 kPa to 40 kPa, from 40 kPa to 50 kPa, from 50 kPa to 60 kPa, from 60 kPa to 70 kPa, from 70 kPa to 80 kPa, from 80 kPa to 90 kPa, from 90 kPa to 100 kPa, from 110 kPa to 120 kPa, from 120 kPa to 130 kPa, from 130 kPa to 140 kPa, from 140 kPa to 150 kPa, from 150 kPa to 160 kPa, from 160 kPa to 170 kPa, from 170 kPa to 180 kPa, from 180 kPa to 190 kPa, from 190 kPa to 200 kPa, from 200 kPa to 210 kPa, from 210 kPa to 220 kPa, from 220 kPa to 230 kPa, from 230 kPa to 240 kPa, from 240 kPa to 250 kPa, from 250 kPa to 300 kPa, from 300 kPa to 350 kPa, from 350 kPa to 400 kPa, from 400 kPa to 450 kPa, from 450 kPa to 500 kPa, from 500 kPa to 550 kPa, from 550 kPa to 600 kPa, from 600 kPa to 650 kPa, or from 650 kPa to 700 kPa. Optionally, an extrusion pressure of the extrusion-based printing method greater than or about 700 kPa. In some examples, the extrusion-based printing method uses a nozzle having a diameter of from 1 μm to 1000 μm, such as from 1 μm to 10 μm, from 10 μm to 100 μm, from 100 μm to 700 μm, from 300 μm to 700 μm, from 100 μm to 200 μm, from 200 μm to 300 μm, from 300 μm to 400 μm, from 400 μm to 500 μm, from 500 μm to 600 μm, from 600 μm to 700 μm, from 700 μm to 800 μm, from 800 μm to 900 μm, or from 900 μm to 1000 μm. Optionally, a temperature of the emulsion during the printing is from about 4° C. to about 50° C., such as from 4° C. to 10° C., from 10° C. to 20° C., from 20° C. to 30° C., from 30° C. to 40° C., or from 40° C. to 50° C. Optionally, printing the emulsion comprises receiving the particles on a surface, wherein the surface has a temperature of about room temperature or less than or about −180° C.

In another aspect, systems are described herein, such as systems for preparing particles (e.g., microparticles and/or nanoparticles), optionally according to the methods described herein. In some examples, a system of this aspect comprises an emulsion supply container for preparing or storing an emulsion; one or more extrusion-based printing nozzles in fluid communication with the emulsion supply container for generating a plurality of droplets of the emulsion including particles, such as having diameters of from 10 nm to 1000 μm; and a collection surface for receiving the plurality of droplets of the emulsion from the one or more extrusion-based printing nozzles. In examples, the emulsion may comprise water, a polymer or a non-polymeric excipient, a solvent, and an active pharmaceutical ingredient. In some examples, the collection surface comprises a sterile vial.

Systems of this aspect can include various components or adjustable parameters to allow for preparing particles (e.g., microparticles and/or nanoparticles), such as according to the methods described herein. For example, systems of this aspect may further comprise one or more mixing vessels in fluid communication with the emulsion supply container for preparing and providing the emulsion to the emulsion supply container. In some examples, the collection surface may optionally be cooled to a temperature of from about −200° C. to about −75° C. A system of this aspect may further comprise a cooling or refrigeration system coupled to the collection surface for cooling the collection surface to a temperature of from about −200° C. to about −75° C. Optionally, a system of this aspect may comprise one or more temperature sensors or temperature controllers for monitoring or controlling a temperature of the collection surface. In some examples, the collection surface is a moving or movable or translating or translatable collection surface. Optionally, a system of this aspect may further comprise a translation stage for generating a relative translation between the one or more extrusion-based printing nozzles and the collection surface. In some examples, a system of this aspect may further comprise one or more pressure sensors or pressure controllers for monitoring or controlling an extrusion pressure associated with the one or more extrusion-based printing nozzles. Optionally, a system of this aspect may further comprise one or more actuators for monitoring or controlling an extrusion speed associated with the one or more extrusion-based printing nozzles.

In some examples, it may be desirable to prepare particles (e.g., microparticles and/or nanoparticles) under sterile conditions. Optionally, a system of this aspect may further comprise a housing for maintaining at least the one or more extrusion-based printing nozzles and the collection surface in a sterile environment. Optionally, a system of this aspect may further comprise sterilization equipment positioned to sterilize one or more of the emulsion supply container (e.g., printing ink container), the one or more extrusion-based printing nozzles, or the collection surface.

In another aspect compositions are provided herein, such as microparticle-based therapeutic compositions. In some examples, a composition may comprise particles having diameters of from 10 nm to 1000 μm; and one or more live cells. In some examples, the particles may have diameters of from about 10 nm to about 1 mm or larger. For example, the particles may have diameters of from 10 nm to 20 nm, from 20 nm to 30 nm, from 30 nm to 40 nm, from 40 nm to 50 nm, from 50 nm to 60 nm, from 60 nm to 70 nm, from 70 nm to 80 nm, from 80 nm to 90 nm, from 90 nm to 100 nm, from 100 nm to 150 nm, from 150 nm to 200 nm, from 200 nm to 250 nm, from 250 nm to 300 nm, from 300 nm to 350 nm, from 350 nm to 400 nm, from 400 nm to 450 nm, from 450 nm to 500 nm, from 500 nm to 600 nm, from 600 nm to 700 nm, from 700 nm to 800 nm, from 800 nm to 900 nm, from 900 nm to 1 μm, from 1 μm to 2 μm, from 2 μm to 3 μm, from 3 μm to 4 μm, from 4 μm to 5 μm, from 5 μm to 6 μm, from 6 μm to 7 μm, from 7 μm to 8 μm, from 8 μm to 9 μm, from 9 μm to 10 μm, from 10 μm to 20 μm, from 20 μm to 30 μm, from 30 μm to 40 μm, from 40 μm to 50 μm, from 50 μm to 60 μm, from 60 μm to 70 μm, from 70 μm to 80 μm, from 80 μm to 90 μm, from 90 μm to 100 μm, from 100 μm to 150 μm, from 150 μm to 200 μm, from 200 μm to 250 μm, from 250 μm to 300 μm, from 300 μm to 350 μm, from 350 μm to 400 μm, from 400 μm to 450 μm, from 450 μm to 500 μm, from 500 μm to 600 μm, from 600 μm to 700 μm, from 700 μm to 800 μm, from 800 μm to 900 μm, or from 900 μm to 1 mm. In some examples, the particles (e.g., microparticles and/or nanoparticles) are attached to surfaces of the one or more live cells. In some examples, the one or more live cells are at least partially encapsulated into the particles. Example live cells include, but are not limited to, live yeast cells, live recombinant yeast cells, live fungal cells, live bacterial cells, live recombinant bacterial cells, live insect cells, live mammalian cells, or live mesenchymal stem cells. Optionally, the particles may be in a lyophilized condition.

Example particles include microparticles or nanoparticles comprising a polymer or a non-polymeric excipient, such as prepared according to various methods described herein or prepared using various systems described herein. Optionally, for particles comprising a polymer, the polymer is a biodegradable polymer selected from the group consisting of poly(lactide-co-glycolide), polylactide (PLA), polyglycolide (PGA), polycaprolactone (PCL), pluronic F127, sodium alginate, hyaluronic acid, chitosan, cyclodextrin, dextran, agarose, gelatin, albumin, collagen, lipids, a polyethylene glycol (PEG) derivative, a pharmaceutical grade polymer, poly(hydroxy butyrate), poly(β-malic acid), or poly(L-lysine). Optionally, for particles comprising a non-polymeric excipient, the non-polymeric excipient is a hydrophilic substance, a hydrophobic substance, a non-reducing sugar, trehalose, sucrose, a polyol, mannitol, sorbitol, xylitol, an amino acid, leucine, or L-arginine.

In some examples, the particles (e.g., microparticles and/or nanoparticles) further comprise an active ingredient embedded within or adsorbed to the particles. For example, the active pharmaceutical ingredient may include one or more of a protein, an antibody, a nucleic acid, messenger ribonucleic acid (mRNA) molecules, a lipid nanoparticle, clustered regularly interspaced short palindromic repeats (CRISPR)-associated protein 9 (Cas9), transcription activator-like effector nucleases (TALENs), zinc-finger nucleases (ZFNs), homing endonucleases or meganucleases, a growth factor, a plasmid, a hydrophilic pharmaceutical, a lipophilic pharmaceutical, a viral particle, a virus-like particle, a live yeast cell, a live recombinant yeast cell, a live fungus, a live bacterial cell, a live recombinant bacterial cell, a live insect cell, a live mammalian cell, or a live mesenchymal stem cell.

Without wishing to be bound by any particular theory, there can be discussion herein of beliefs or understandings of underlying principles relating to the invention. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1 provides a schematic illustration showing different routes for preparing an emulsion for microparticle generation.

FIG. 2 provides a schematic illustration showing the generation of microparticles using an extrusion-based process and summarizing different microparticle formulations that can be prepared using the technique.

FIG. 3A and FIG. 3B provide schematic illustrations showing printing of microparticles using an extrusion-based process directly onto a cooled moving surface assisted by an integrated flash freezing process.

FIG. 4 provides a schematic illustration showing emulsion generation and semi-continuous printing of microparticles directly into collection vials.

FIG. 5 shows differential scanning calorimetry analysis results for various microparticles and mixtures.

FIG. 6 shows the results of a size distribution analysis for chloroquine-loaded microparticles.

FIG. 7 shows scanning electron micrograph images of chloroquine-loaded microparticles.

FIG. 8 shows the results of a size distribution analysis for 6-thioguanine-loaded microparticles.

FIG. 9 shows scanning electron micrograph images of 6-thioguanine-loaded microparticles (samples were stored at 4° C. for a few days before taking the images).

FIG. 10 shows results of a size distribution analysis for microparticles prepared similarly to the chloroquine-loaded microparticles analyzed for FIG. 6 but without including chloroquine.

FIG. 11 shows results of a size distribution analysis for microparticles prepared similarly to the 6-thioguanine-loaded microparticles analyzed for FIG. 6 but without including 6-thioguanine.

FIG. 12 shows scanning electron micrograph images of microparticles prepared similarly to the chloroquine-loaded microparticles depicted in FIG. 7 but without including chloroquine.

FIG. 13 shows scanning electron micrograph images of microparticles prepared similarly to the 6-thioguanine-loaded microparticles depicted in FIG. 9 but without including 6-thioguanine (samples were stored at 4° C. for a few days before taking the images).

FIG. 14 shows the scanning electron micrograph images of bovine serum albumin (BSA) loaded poly (lactide-co-glycolide) (PLGA) microparticles made on dry ice/cryoprotectant.

FIG. 15 shows the scanning electron micrograph images of bovine serum albumin (BSA) loaded poly (lactide-co-glycolide) (PLGA) microparticles made on liquid nitrogen/cryoprotectant.

FIG. 16 shows scanning electron micrograph images of the ovalbumin (OVA)-loaded poly(lactide-co-glycolide) microparticles.

FIG. 17 shows a circular dichroism (CD) spectrum of free unprocessed ovalbumin (OVA) and the encapsulated ovalbumin (OVA).

FIG. 18 shows Fourier-transform infrared spectrums of free unprocessed ovalbumin (OVA) and the lyophilized ovalbumin (OVA) encapsulated poly (lactide-co-glycolide) (PLGA) microparticles.

FIG. 19 shows differential scanning calorimetry of free unprocessed ovalbumin (OVA) compared with ovalbumin (OVA) loaded poly (lactide-co-glycolide) (PLGA) microparticles and blank poly (lactide-co-glycolide) (PLGA) microparticles.

FIG. 20 shows fluorescent images of ovalbumin (OVA)-fluorescein isothiocyanate (FITC) loaded poly (lactide-co-glycolide) (PLGA) microparticles.

FIG. 21A, FIG. 21B, and FIG. 21C show the ovalbumin (OVA) encapsulation efficiency (EE %) based on various factors.

FIG. 22 shows scanning electron micrograph images of the ovalbumin (OVA) loaded chitosan (CS) microparticles at various magnifications.

FIG. 23A and FIG. 23B show a characterization of ovalbumin (OVA) loaded chitosan (CS) microparticles made with various operational conditions.

FIG. 24 shows a schematic illustration of the fabrication of drug-loaded microparticles using double emulsion evaporation and extrusion-based printing technique.

FIGS. 25A-25B show a schematic representation of (FIG. 25 A) three-factor/two-level factorial design and (FIG. 25 B) face-centered CCD.

FIG. 26A, FIG. 26B, FIG. 26C, FIG. 26D, FIG. 26E, and FIG. 26F show scanning electron micrograph images of 6-TG loaded microparticles fabricated under various printing parameters (the images were captured immediately after the samples were freshly made).

FIGS. 27A-27C show (FIG. 27A) initial Pareto chart with all terms, (FIG. 27B) Pareto chart without 4-way interaction term, and (FIG. 27C) final Pareto chart without terms that are statistically not significant.

FIGS. 28A-28D show (FIG. 28A) normal probability plot of residuals, (FIG. 28B) residuals versus fits plot, (FIG. 28C) histogram of residuals, and (FIG. 28D) residuals versus order plot for four-factor/two-level full factorial design.

FIGS. 29A-29B shows (FIG. 29A) main effects plot and (FIG. 29B) interaction plot for four-factor/two-level full factorial design.

FIGS. 30A-30D show (FIG. 30A) normal probability plot of residuals, (FIG. 30B) residuals versus fits plot; (FIG. 30C) histogram of residuals, and (FIG. 30D) residuals versus order plot for three-factor/two-level full factorial design with center points.

FIGS. 31A-31B show (FIG. 31A) main effects plot and (FIG. 31B) interaction plot for three-factor/two-level full factorial design with center point.

FIGS. 32A-32F show (FIG. 32A) surface plot of DLE versus printing speed and printing pressure, (FIG. 32B) surface plot of DLE versus drug amount and printing speed, (FIG. 32C) surface plot of DLE versus drug amount and printing pressure, (FIG. 32D) contour plot of DLE versus drug amount and printing speed, (FIG. 32E) contour plot of DLE versus drug amount and printing pressure, and (FIG. 32F) contour plot of DLE versus printing speed and printing pressure.

FIGS. 33A-33D show drug release profile of microparticles under different (FIG. 33A) printing pressures (100 kPa and 200 kPa), (FIG. 33B) printing speeds (10 mm/s and 20 mm/s), (FIG. 33C) initial drug inputs (6 mg and 9 mg), and (FIG. 33D) inhibition effect on SARS-CoV PLpro of 6-TG loaded microparticles formulated under different process parameters.

FIG. 34A, FIG. 34B, FIG. 34C, FIG. 34D, and FIG. 34E show scatter plots of predicted values calculated by different machine learning models versus experimental values on the training and test subset.

FIGS. 35A-35D show scatter plots of predicted values calculated by (FIG. 35A) four-factor/two-level full factorial design model, (FIG. 35B) face-centered CCD model, (FIG. 35C) DT model, and (FIG. 35D) RF model versus experimental values on the validation dataset.

FIG. 36 feature importance in DLE prediction ranked by DT algorithm.

### DETAILED DESCRIPTION

Techniques described herein include those employing pneumatic, pressure assisted, extrusion-based 3D printing and emulsion evaporation for fabricating microparticle-based drug delivery systems encapsulating an active pharmaceutical ingredient for the treatment of different diseases. The techniques provide for encapsulation of a variety of substances including proteins, plasmid DNA, lipophilic pharmaceutical compositions, hydrophilic pharmaceutical compositions, live cells, and/or cellular components into polymeric particles. As used throughout, unless otherwise noted, the terms particles and microparticles may also include nanoparticles. A variety of biocompatible polymers can be used to formulate the particles, such as, but not limited to, poly (lactide-co-glycolide) (PLGA), polylactide (PLA), polycaprolactone (PCL), etc. Similarly, various non-polymeric excipients such as non-reducing sugars, such as trehalose, sucrose, or polyols (e.g., mannitol, sorbitol, xylitol), or amino acids (e.g., leucine) can be used as suitable carrier matrices to form the particles.

In some examples, described for purposes of illustrating the printing process in a general sense, an active pharmaceutical ingredient (if hydrophilic) can be dissolved in a poly(vinyl alcohol) (PVA) or other suitable polymeric aqueous solution including but not limited to, polyethylene glycol (PEG) or polyvinyl pyrrolidone (PVP), or lipidic solutions, optionally with a cosolvent to aid in dissolution in the case of hydrophobic active pharmaceutical ingredients. The active pharmaceutical ingredient dissolved in the aqueous solution, such as PVA solution, can be further added to a polymer dissolved in an organic solvent that may be immiscible or partially miscible with water, such as chloroform, followed by mixing completely to form a primary emulsion. The primary emulsion can be further added to another aqueous solution, such as PVA with an optionally higher PVA concentration, followed by mixing completely to generate a secondary emulsion. The secondary emulsion can be transferred to a pneumatic syringe with a fine gauge needle, then printed by a bioprinter employing an extrusion-based printing step. After the evaporation of the organic solvent, the resultant particles can optionally be washed (e.g., by ultracentrifugation) one or more times, and then collected.

In precipitation based methods, the organic solvent may be miscible with water. The organic phase may mostly contain the polymeric carrier and the payload, and the water phase may or may not contain a surfactant or stabilizer, such as PVA. In chitosan gelation, chitosan or any other polymer that has intermolecular interactions (such as electrostatic or hydrophobic interactions) with the payload can be mixed with it at certain volume ratio. In presence of a crosslinker (e.g., adding an ionic crosslinker such as calcium chloride or sodium sulfate or heating the nozzle up for thermal gelation) the polymer may form a gel and encapsulates the payload in it. Extrusion may break the gel into smaller particles.

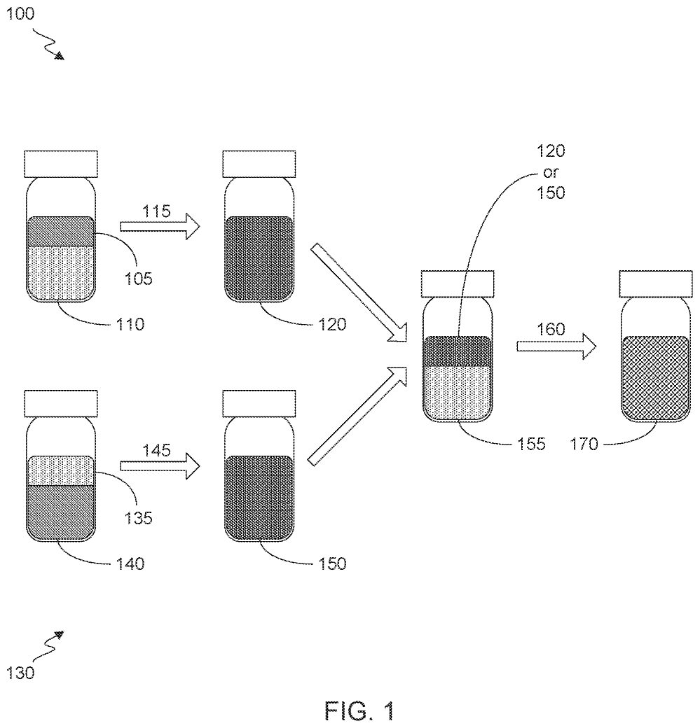

FIG. 1 provides a schematic overview of processes 100 and 130 of preparing emulsions used in the techniques described herein. Process 100 includes combining a hydrophobic active ingredient (e.g., a pharmaceutical compound) and a polymer in an organic phase 105 and an aqueous phase 110 and mixing 115 to generate an emulsion 120, such as an oil-in-water emulsion. Process 130 includes combining a polymer in an organic phase 135 and a hydrophilic active ingredient (e.g., a pharmaceutical compound) in an aqueous phase 140 and mixing 145 to generate an emulsion 150, such as a water-in-oil emulsion. Emulsion 120 or emulsion 150 can then be combined with an additional aqueous phase 155 and mixed 160 to generate a water-in-oil-in-water emulsion 170. Emulsion 170 is suitable for generating particles of the active ingredients embedded in the polymer using extrusion-based printing techniques described herein. Advantageously, emulsion 170 can be prepared without the use of sonication or other ultrasonic mixing techniques that can result in raising the emulsion temperature, allowing for temperature sensitive active ingredients to be incorporated into polymeric particles without being subjected to excessive temperatures. Organic phase 105 or organic phase 135 may be a solvent and subject to evaporation, in some cases.

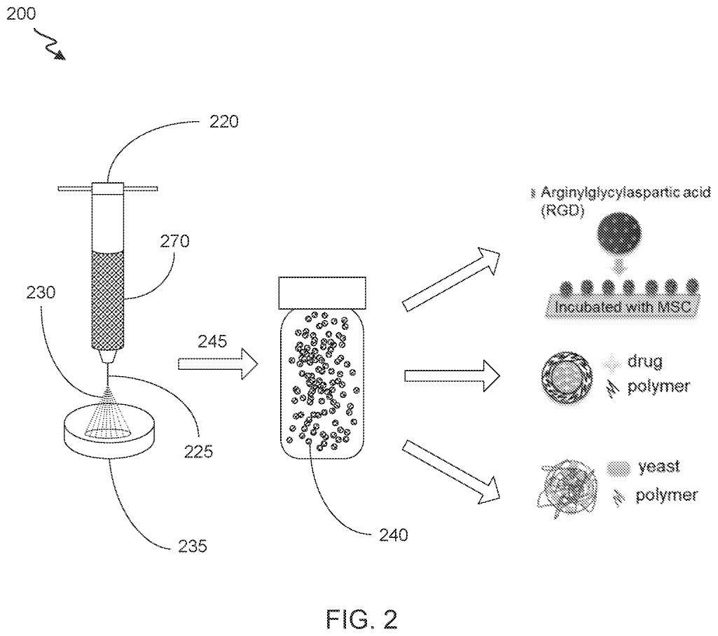

FIG. 2 provides a schematic overview of a process 200 of generating particles (e.g., microparticles and/or nanoparticles) from an emulsion 270. Emulsion 270 may be the same as or different from emulsion 170, but may comprise an emulsion suitable for preparing particles using a combination of extrusion-based printing and solvent evaporation. Process 200 shows emulsion 270 in a syringe 220, where the emulsion is extruded through syringe needle 225 in an extrusion process. Syringe 220 may be or comprise a syringe pump or other component of a bioprinter or other extrusion-based printing system. The process of forcing the emulsion 270 through syringe needle 225 subjects the emulsion 270 to shear stresses that cause the emulsion to break up into droplets 230 containing particles 235. The droplets 230 and particles 235 can be collected in a suitable collection vessel 240, which may be a petri dish, sample vial, or the like and may be in a sterile condition to prevent contamination of the particles 240. Particles 240 may be subjected to a series of additional processing steps 245, such as a solvent evaporation step and a washing step (e.g., by centrifugation), to remove excess material from droplets 230, such as excess solvent, excess polymer, excess active ingredients, or the like. Particles 240, after solvent evaporation and washing, may be collected into a suitable collection vessel for storage and later use. FIG. 2 shows some example formulations for particles 240.

During the SMART processing of blank and drug-loaded particles (e.g., microparticles and/or nanoparticles), various process parameters are useful for impacting particle characteristics. For example, these process parameters may be tuned to generate particles with a size distribution ranging from 10 nm to 2 μm. The printing parameters include, but are not limited to, extrusion pressure (e.g., about 200 kPa), nozzle diameter, such as from 18 to 34 gauge, and micro-S (e.g., 50, 100, or 150 μm). Another process parameter may include the temperature of the mixture/emulsion, which may range from 30° C. to 60° C., for example. Another process parameter may include a print bed temperature, which may ranging from 4° C. to 60° C. (e.g., when under ambient conditions) for standard operation or at −80° C. or below (e.g., less than or about −196° C.) for cryogenic operation. Another process parameter may include a weight ratio of active ingredient to polymer (e.g., about 1:10), and a stabilizer concentration (e.g., about 8%).

As one example, the particles 240 may be used for a combination of particulate-based drug delivery and stem cell therapy. For example, the particles 240 may comprise Arginylclycylaspartic acid (RGD) and can be attached to or incubated with mesenchymal stem cells (MSC), which can not only aid in navigating particles to the site of action for disease treatment but also promote the recovery of damaged tissue/organs.

As another example, the particles 240 may comprise a polymer, such as a biodegradable polymer (e.g., PLGA), and a pharmaceutical drug encapsulated by the polymer. The particles 230 can be administered to a subject in need of the pharmaceutical drug.

As another example, the particles 240 may comprise a polymer, such as a biodegradable polymer (e.g., PLGA), and live cells, such as yeast cells (e.g., Pichia pastoris) or bacterial cells (e.g., Escherichia coli). The cells encapsulated within the polymeric particles 240 can be used for antimicrobial or probiotic applications, for example. In some cases, the cells may be genetically modified, for example, to produce recombinant proteins, such as for use in or as a vaccine. The particles 240 with the live cells can optionally be subjected to a lyophilization (freeze drying) process, allowing the particles 240 to be stored for prolonged durations and later reconstituted and used as an inoculant for later cell growth and preparation of the recombinant proteins, when needed.

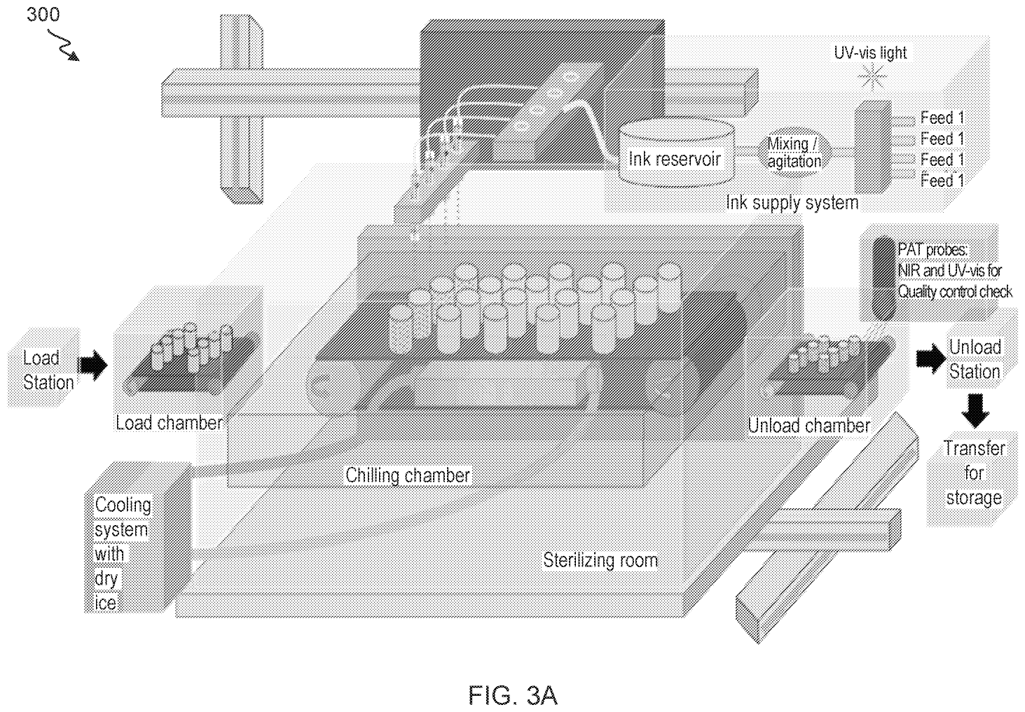

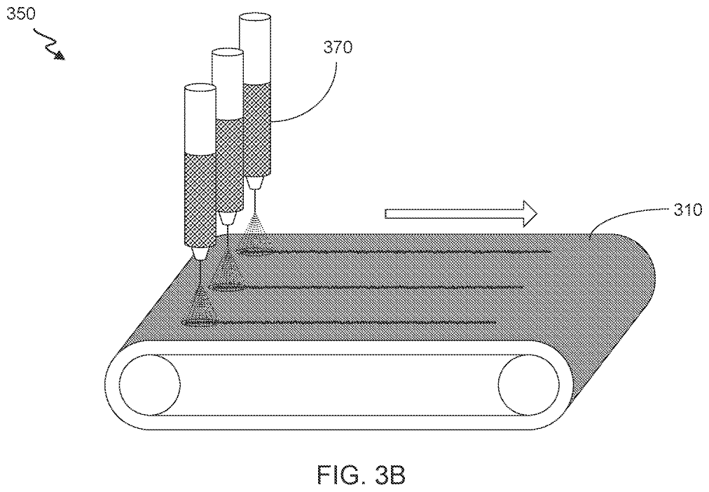

In some cases, particles (e.g., microparticles and/or nanoparticles) can be prepared using continuous or semi-continuous printing processes. FIGS. 3A and 3B show schematic illustrations of example systems 300 and 350 for preparation of lyophilized particles, such as using a thin film freeze drying process in a continuous or semi-continuous manner. As illustrated, an emulsion 370 can be subjected to an extrusion-based printing technique, as described above, to generate droplets containing particles that are deposited directly onto a pre-cooled cryogenic surface 310 (e.g., in the form of vials placed on a moving surface, as in FIG. 3A, or a conveyor, as in FIG. 3B), leading to their ultra-rapid freezing (e.g., at a cooling rate of 100-1000 K/s) in the form of frozen particles. This process allows the components to freeze uniformly with a consistent rate, in contrast to bulk freezing processes, preventing nucleation and preserving the structure of the components. The droplets can optionally be collected in a non-reactive cryogenic liquid (e.g., liquid nitrogen), and the frozen solvents of the droplet thin films can be evaporated in a lyophilizer for an appropriate amount of time. FIG. 3A shows details for example system 300, including an ink supply system, chambers and areas for loading and unloading vials, a sterilization room, a cooling system, and translation stages connected to at least the extrusion nozzles and the sterilizing room.

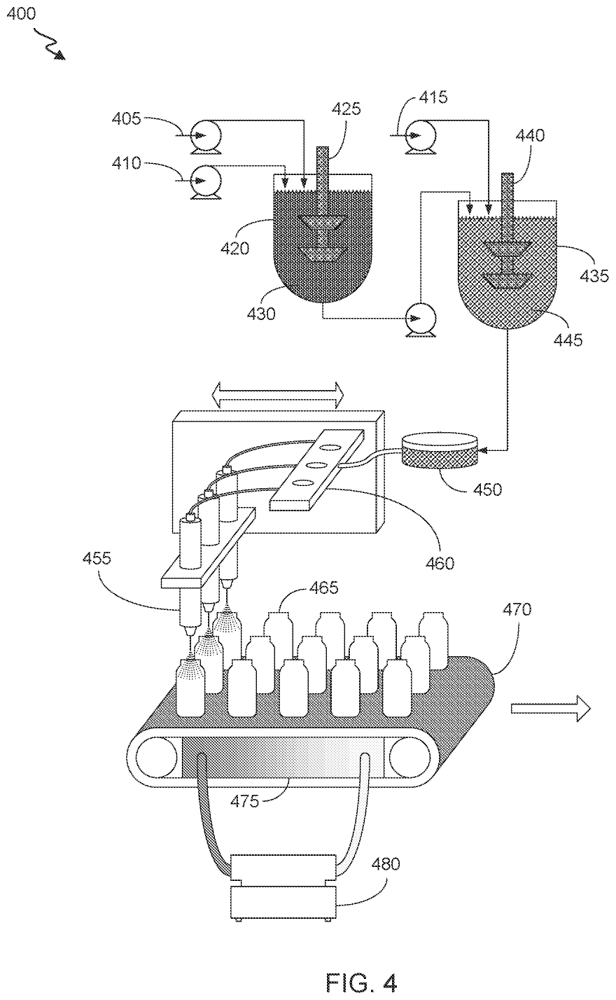

FIG. 4 provides a schematic illustration of an example system 400 for preparing particles (e.g., microparticles and/or nanoparticles) in a continuous or semi-continuous process, including preparation of an emulsion for use in an extrusion-based printing and solvent evaporation process. System 400 includes a plurality of sources, which may correspond to an aqueous phase source 405, an organic phase source 410, and an outer aqueous phase source 415. The aqueous phase source 405 and the organic phase source 410 can provide, for example via pumps, an aqueous solution and an organic solution, such as including polymers and active ingredients as described above with reference to FIG. 1, to a mixing vessel 420 with an agitation system 425. Agitation system 425 can mix the aqueous solution and organic solution to form a primary emulsion 430, which can comprise an oil-in-water emulsion (e.g., emulsion 120) or a water-in-oil emulsion (e.g., emulsion 150). Primary emulsion 430 can be provided to a second mixing vessel 435 with an agitation system 440, and combined with an aqueous solution from outer aqueous phase source 415, such as via pumps. Agitation 440 can mix primary emulsion 430 and the aqueous solution to form a secondary emulsion 445.

Secondary emulsion 445 can be directed to a reservoir 450 for distribution to a plurality of extrusion heads 455, such as via a manifold 460. Extrusion heads can comprise syringe pumps, for example. In some cases, extrusion heads 455, and optionally manifold 460 and/or reservoir 450, can be movable in a lateral direction to allow adjustment of the position of the extrusion heads 455 to extrude droplets containing microparticles into an array of collection vials 465, which may be sterile. In some cases, collection vials 465 may be on a conveyor 470 or other translation stages, allowing translation of collection vials 465 relative to extrusion heads 455. As illustrated, a conveyor may 470 be used to transport collection vials 465 out of system 400 when filled, such as in a batch process or on a continuous or semi-continuous basis. Conveyor 470 may include a chilling block 475, with coolant provided by an inlet and an outlet to a refrigeration or other cooling system 480, such as a dry-ice-based cooling system or a liquid-nitrogen-based cooling system. Chilling block 475 may allow for collection vials 465 to be at cryogenic or ultra-low temperatures (e.g., temperatures of −75° C. or less, such as from −75° C. to −200° C.). Such as system 400 can allow for directly printing particles in a solid frozen condition in a sterile collection vial in a single step. The filled collection vials 165 can be transported and stored for lyophilization and any additional desired processing.

In one case, dry ice was used to maintain an ultra-low temperature at the printing bed/substrate (e.g., about −80° C.), system 400 can allow for directly printing particles in a collection vial with a specially designed collection (umbrella-shaped) lid on the top. In another case, liquid nitrogen was used to cool the collection vial down to about −178° C. (or close to −196° C.), then system 400 directly printed into the ultra-cool vial maintained at the ultra-low temperature. Example 4 describes the details. This process printed particles directly onto ultra-low temperature surface that ensured flash freezing the particles into solid forms which were subjected to further drying process such as lyophilization to obtain dry solid particles (e.g., dry solid microparticles and/or dry solid nanoparticles).

In some cases, system 400 or various components thereof may be enclosed in a sterile chamber, allowing preparation and filling collection vessels 465 with particles under sterile conditions. In some cases, sterilizing equipment may be included in system 400, such as ultraviolet lamps. Optionally, system 400 may include various process analytical technologies, such as near-infrared (NIR) or ultraviolet (UV)-visible (VIS) probes, to provide inline monitoring functionalities, such as to monitor the quality of the source materials (e.g., aqueous solutions, organic solutions, etc.), of the primary or secondary emulsions, or of the printed particles.

System 400 may optionally include one or more pressure sensors or pressure controllers, temperature sensors or temperature controller, position sensors, translation stages,

The invention may be further understood by the following non-limiting examples.

In some of the below Examples, PLGA is used as the polymer to fabricate particles encapsulating pharmaceutical agents, 6-thioguanine (6-TG) and chloroquine, useful for COVID-19 treatment, for example. 6-TG is a water-insoluble drug, which dissolves in NaOH solution, so the addition of NaOH aims to improve the solubility as a cosolvent. Chloroquine is a hydrophilic drug that can easily dissolve in a 1% poly(vinyl alcohol) (PVA) solution. The fabricated particles are suitable for particulate-based drug delivery platforms.

Outlining the mechanism, the process in these Examples involves combining an emulsion evaporation method and an extrusion-based printing method. Once prepared, a secondary emulsion is transferred to a pneumatic syringe, and the shear stress exerted by syringe nozzle is employed to generate micro-sized emulsion droplets. After the evaporation of chloroform, the emulsion droplets are solidified to generate drug-loaded particles. A washing step by ultracentrifugation removes unencapsulated active pharmaceutical ingredients and residual PVA. The addition of NaOH results in the porous surface structure of 6-TG-loaded particles, allowing the attachment of cells if desired.

### Example 1

Poly(lactide-co-glycolide) (PLGA) microparticles loaded with chloroquine were prepared according to the following process. Table 1 shows the formulation used to prepare these chloroquine-loaded microparticles.

As the inner aqueous phase, chloroquine was added to 0.5 ml of a poly(vinyl alcohol) (PVA) solution (1% w/w). 30 mg of PLGA was dissolved in 2 ml of chloroform to form the organic phase. The inner aqueous phase was added into the organic phase and the mixture was then mixed to obtain a homogenous primary emulsion. The primary emulsion was then transferred into 8 ml of a 2% PVA solution, followed by mixing completely to generate a secondary emulsion. The secondary emulsion was transferred to a 3 ml pneumatic syringe with a 25-gauge needle, then printed by a 3D bioprinter (extrusion-based printing) with a printing speed of 20 mm/s, under a pressure of 200 kPa. After the evaporation of organic solvent, the chloroquine-loaded microparticles were separated and washed by ultracentrifugation twice. After the washing step, the chloroquine-loaded microparticles were collected and characterized using various characterization techniques.

The yield, encapsulation efficiency, and chloroquine loading were evaluated. The process exhibited a high yield of chloroquine-loaded microparticles, ≥86.1%. The total encapsulation efficiency was determined to be close to 100%, such as >17.09±2.61% (encapsulated in the polymeric carrier) and >80% adsorbed into the polymeric particles, and the loading was determined to be >1.76±0.27% (w/w).

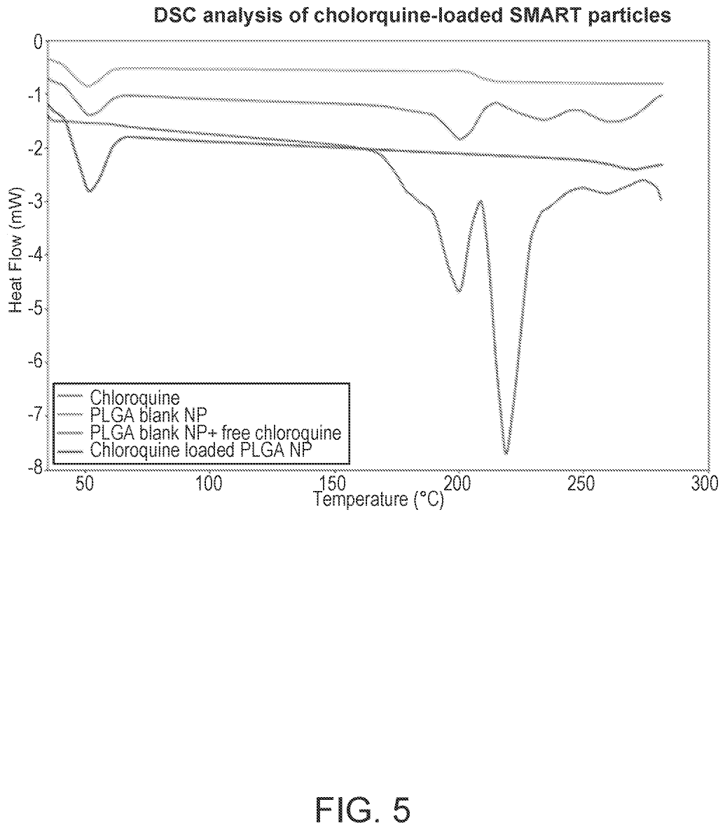

The chloroquine-loaded microparticles were subjected to thermal characterization using differential scanning calorimetry. FIG. 5 shows a plot of temperature versus heat flow for the chloroquine-loaded microparticles and compares with the thermal characterization of chloroquine by itself, blank microparticles (for chloroquine) prepared according to Example 3 mixed with free chloroquine, and blank microparticles (for chloroquine) prepared according to Example 3.

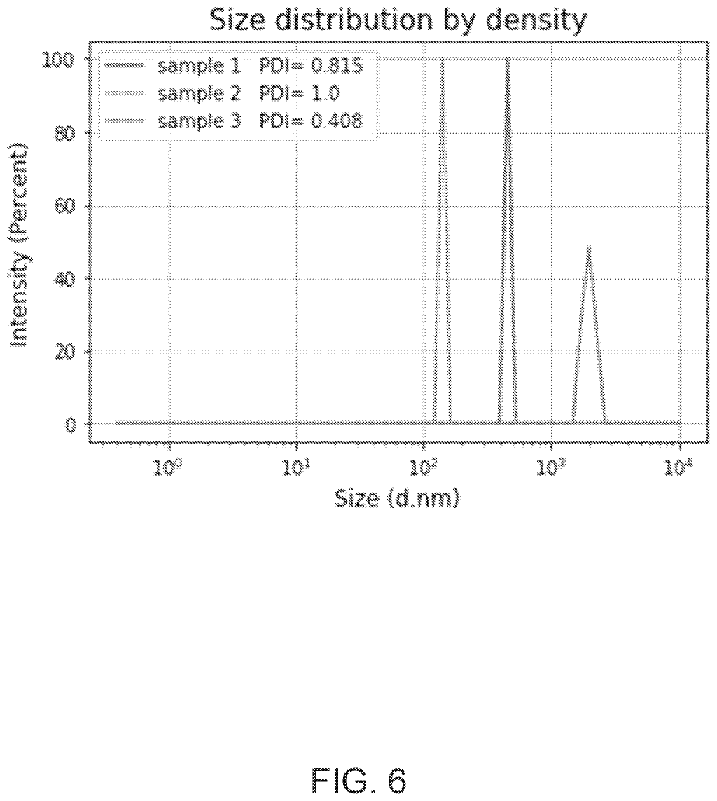

Multiple samples of the chloroquine-loaded microparticles were prepared according to the above procedures and the size distributions of the different samples of the chloroquine-loaded microparticles were measured. FIG. 6 shows the size distribution results for the chloroquine-loaded microparticles, with the polydispersity index (PDI) shown in the legend, indicating that highly monodisperse and highly polydisperse groups of microparticles can be prepared.

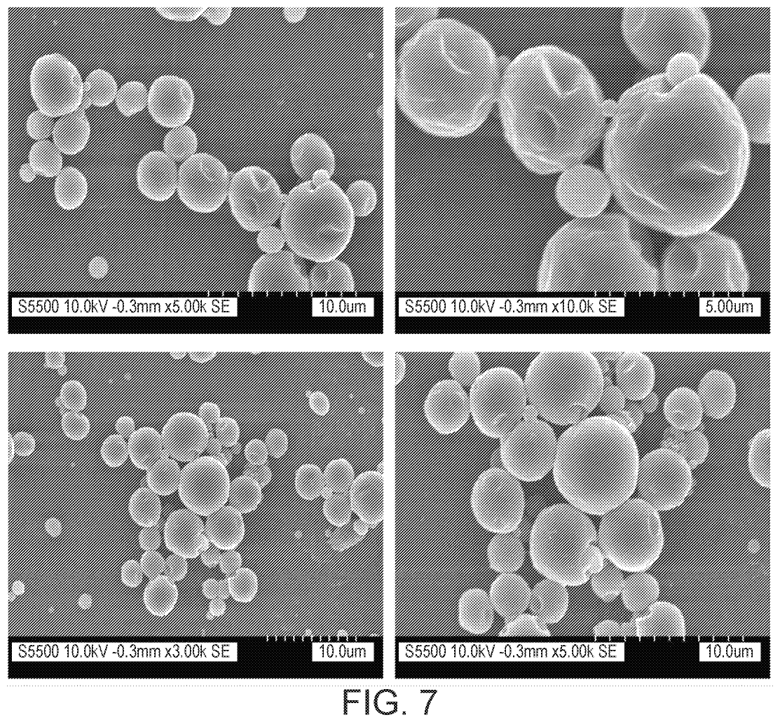

Samples of the chloroquine-loaded microparticles were subjected to imaging using scanning electron micrography (SEM). Images of chloroquine-loaded microparticles are shown in FIG. 7.

TABLE 1
Formulation for chloroquine-loaded microparticles

| | | Amount | |
|---|---|---|---|
| Ingredient | Purpose | per batch | |
| Chloroquine | active pharmaceutical | 3 | mg |
| | ingredient | | |
| PLGA | Polymer | 30 | mg |
| PVA | Surfactant | 160 | mg |
| Chloroform | Organic solvent | 2 | ml |

### Example 2

Poly(lactide-co-glycolide) (PLGA) microparticles loaded with 6-thioguanine (6-TG) were prepared according to the following process. Table 2 shows the formulation used to prepare these 6-TG-loaded microparticles.

As the inner aqueous phase, 6-TG was dissolved in 0.5 ml of a 0.11 M NaOH poly(vinyl alcohol) (PVA) solution (1% w/w). 30 mg of PLGA was dissolved in 2 ml of chloroform to form the organic phase. The inner aqueous phase was added into the organic phase and the mixture was then mixed to obtain a homogenous primary emulsion. The primary emulsion was then transferred into 8 ml of a 2% PVA solution, followed by mixing completely to generate a secondary emulsion. The secondary emulsion was transferred to a 3 ml pneumatic syringe with a 25-gauge needle, then printed by a 3D bioprinter (extrusion-based printing) with a printing speed of 20 mm/s, under a pressure of 200 kPa. After the evaporation of the organic solvent, the 6-TG-loaded microparticles were separated and washed by ultracentrifugation twice. After the washing step, the 6-TG-loaded microparticles were collected and characterized using various characterization techniques.

The yield, encapsulation efficiency, and 6-TG loading were evaluated. The process exhibited a high yield of 6-TG-loaded microparticles, about 86.1%. The encapsulation efficiency was determined to be about 100%, (about 63% encapsulated and about 37% adsorbed to or around the surface of the particles), and the loading was determined to be >6.58±0.03% (w/w).

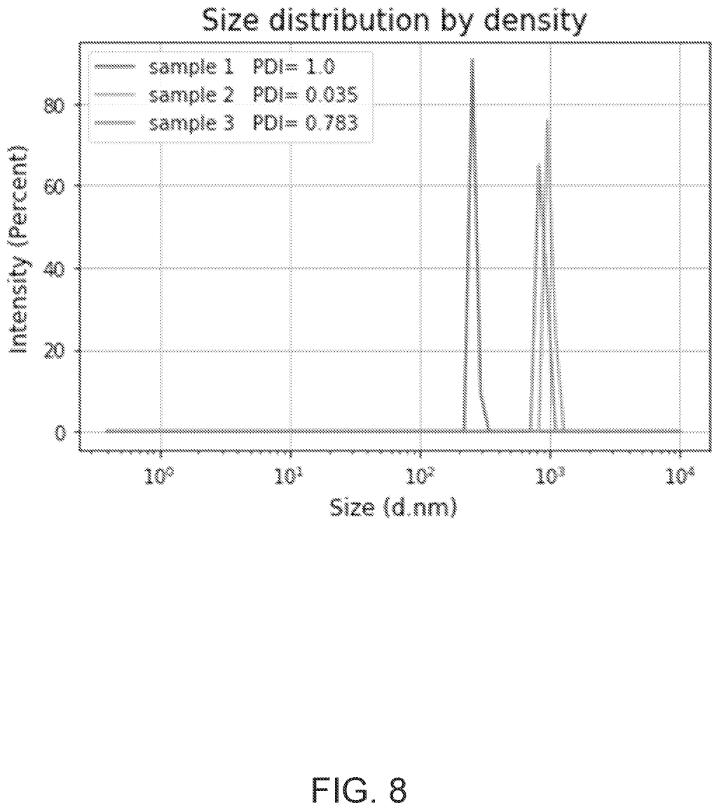

Multiple samples of the 6-TG-loaded microparticles were prepared according to the above procedures and the size distributions of the different samples of the 6-TG-loaded microparticles were measured. FIG. 8 shows the size distribution results for the 6-TG-loaded microparticles, with the polydispersity index (PDI) shown in the legend, indicating that highly monodisperse and highly polydisperse groups of microparticles can be prepared.

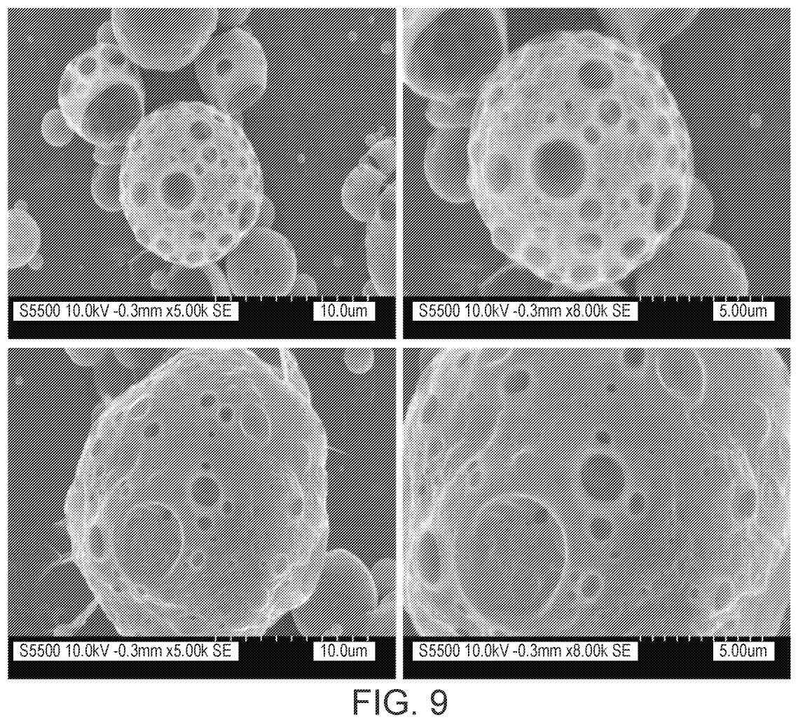

Samples of the 6-TG-loaded microparticles were subjected to imaging using scanning electron micrography (SEM). SEM images of 6-TG-loaded microparticles are shown in FIG. 9, and these particles show a much more porous character than the chloroquine-loaded microparticles shown in FIG. 7, due to the use of NaOH as a cosolvent.

TABLE 2
Formulation for 6-TG-loaded microparticles

| | | Amount | |
|---|---|---|---|
| Ingredient | Purpose | per batch | |
| 6-TG | active pharmaceutical | 3 | mg |
| | ingredient | | |
| NaOH | Cosolvent | 2.4 | mg |
| PLGA | Polymer | 30 | mg |
| PVA | Surfactant | 165 | mg |
| Chloroform | Organic solvent | 2 | ml |

### Example 3

Poly(lactide-co-glycolide) (PLGA) microparticles without any active pharmaceutical ingredient (“blank microparticles”) were prepared according to the following process.

A first group of blank microparticles was prepared using the same process as the chloroquine loaded microparticles as described above in Example 1, except that the chloroquine was not used. The resultant blank microparticles are referred to herein as “blank microparticles (for chloroquine)”.

As the inner aqueous phase, 0.5 ml of a poly(vinyl alcohol) (PVA) solution (1% w/w) was used. 30 mg of PLGA was dissolved in 2 ml of chloroform to form the organic phase. The inner aqueous phase was added into the organic phase and the mixture was then mixed to obtain a homogenous primary emulsion. The primary emulsion was then transferred into 8 ml of a 2% PVA solution, followed by mixing completely to generate a secondary emulsion. The secondary emulsion was transferred to a 3 ml pneumatic syringe with a 25-gauge needle, then printed by a 3D bioprinter (extrusion-based printing) with a printing speed of 20 mm/s, under a pressure of 200 kPa. After the evaporation of the organic solvent, the blank microparticles (for chloroquine) were separated and washed by ultracentrifugation twice. After the washing step, the blank microparticles (for chloroquine) were collected and characterized using various characterization techniques. The process exhibited a high yield of blank microparticles (for chloroquine), about 91.5%.

The blank microparticles (for chloroquine) were subjected to thermal characterization using differential scanning calorimetry. FIG. 5 shows a plot of temperature versus heat flow for the blank microparticles (for chloroquine), and compares with the thermal characterization of chloroquine by itself, the blank microparticles (for chloroquine) mixed with free chloroquine, and chloroquine loaded microparticles prepared according to Example 1 above.

A second group of blank microparticles was prepared using the same process as the 6-thioguanine (6-TG) loaded microparticles as described above in Example 2, except that the 6-TG was not used. The resultant blank microparticles are referred to herein as “blank microparticles (for 6-TG)”.

As the inner aqueous phase, 0.5 ml of a 0.11 M NaOH poly(vinyl alcohol) (PVA) solution (1% w/w) was used. 30 mg of PLGA was dissolved in 2 ml of chloroform to form the organic phase. The inner aqueous phase was added into the organic phase and the mixture was then mixed to obtain a homogenous primary emulsion. The primary emulsion was then transferred into 8 ml of a 2% PVA solution, followed by mixing completely to generate a secondary emulsion. The secondary emulsion was transferred to a 3 ml pneumatic syringe with a 25-gauge needle, then printed by a 3D bioprinter (extrusion-based printing) with a printing speed of 20 mm/s, under a pressure of 200 kPa. After the evaporation of the organic solvent, the blank microparticles (for 6-TG) were separated and washed by ultracentrifugation twice. After the washing step, the blank microparticles (for 6-TG) were collected and characterized using various characterization techniques. The process exhibited a high yield of blank microparticles (for 6-TG), about 94.5%.

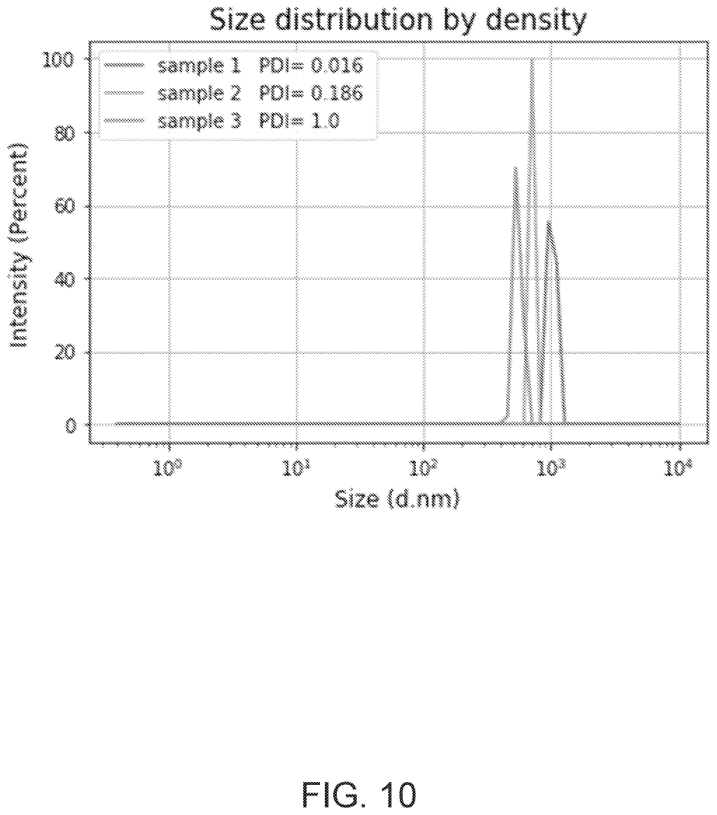

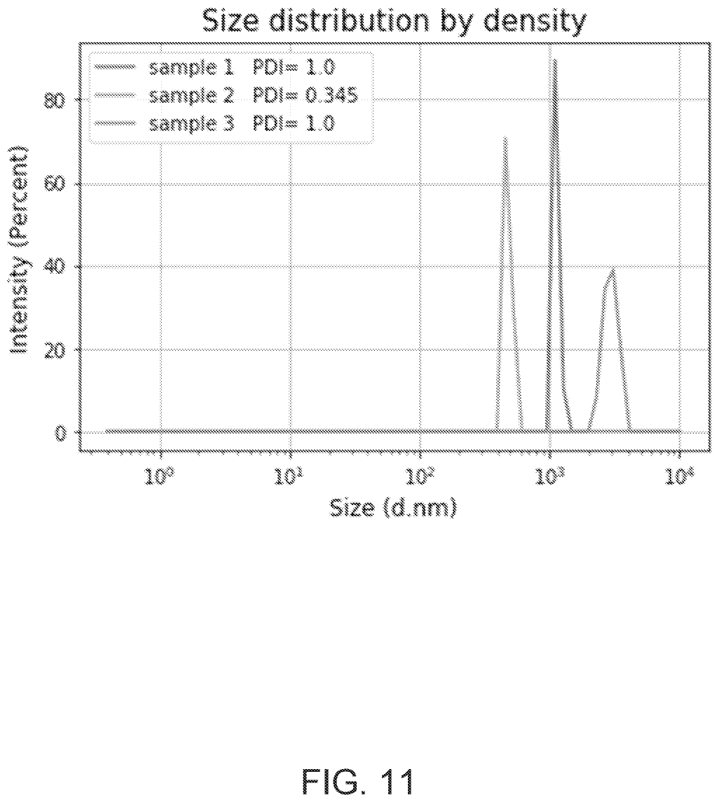

Multiple samples of the blank microparticles were prepared according to the above procedures and the size distributions of the different samples of the blank microparticles were measured. FIG. 10 shows the size distribution results for blank microparticles (for chloroquine) and FIG. 11 shows the size distribution results for blank microparticles (for 6-TG), with the polydispersity index (PDI) shown in the legends, indicating that highly monodisperse and highly polydisperse groups of microparticles can be prepared.

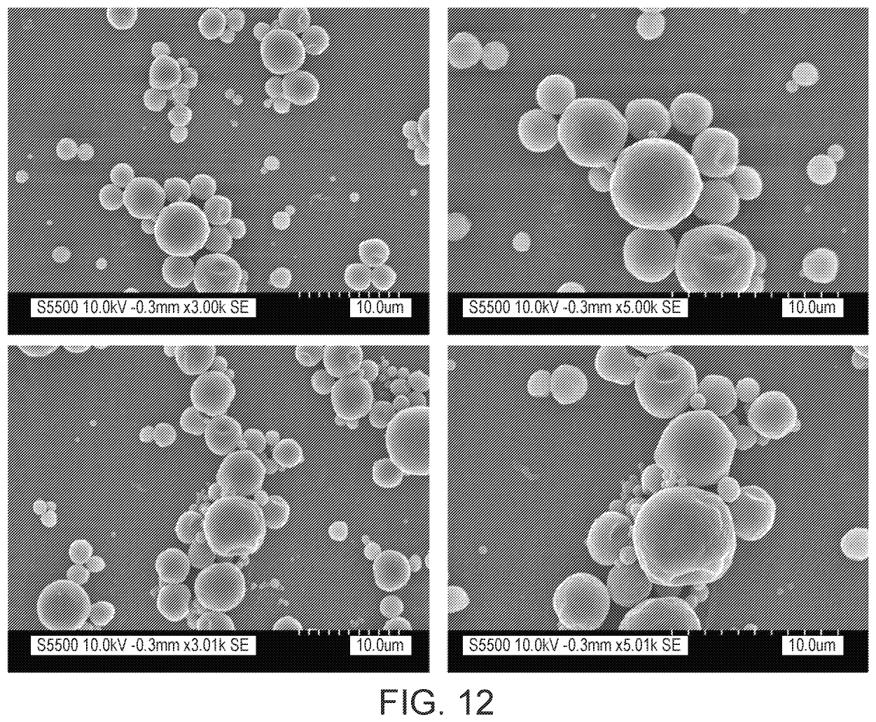

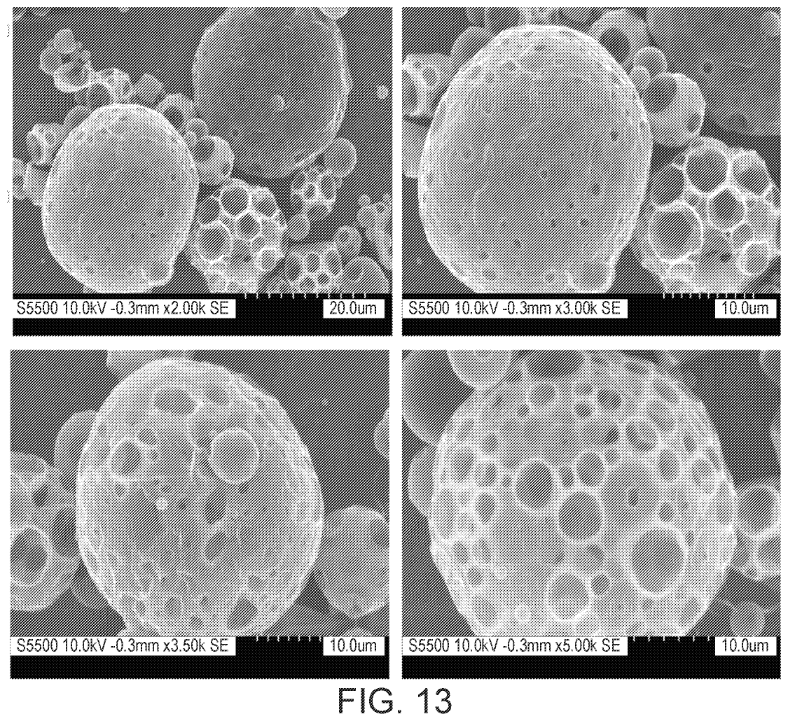

Samples of the blank microparticles were subjected to imaging using scanning electron micrography (SEM). Images of blank microparticles (for chloroquine) are shown in FIG. 12 and images of blank microparticles (for 6-TG) are shown in FIG. 13. The blank microparticles (for chloroquine) appeared to be quite similar to the chloroquine-loaded microparticles of Example 1, and the blank microparticles (for 6-TG) appeared to be quite similar to the 6-TG-loaded microparticles of Example 2.

### Example 4

Poly(lactide-co-glycolide) (PLGA) microparticles loaded with bovine serum albumin (BSA) were prepared according to the following process. Table 3 shows the formulation used to prepare these BSA-loaded microparticles.

As the inner aqueous phase, BSA was dissolved in 0.5 ml of a poly(vinyl alcohol) (PVA) solution (1% w/w). 30 mg of PLGA was dissolved in 2 ml of chloroform to form the organic phase. The inner aqueous phase was added into the organic phase and gently mixed by to obtain a homogenous primary emulsion. The primary emulsion was then transferred into 8 ml of a 2% PVA solution containing 2.5% sucrose, followed by gently mixing to generate a secondary emulsion.

The secondary emulsion was transferred to a 3 ml pneumatic syringe with a 25-gauge needle, then printed into a vial containing dry ice (−80° C.) by a 3D bioprinter (extrusion-based printing) with a printing speed of 1 mm/s, under a pressure of 200 kPa. The flash frozen BSA-loaded PLGA microparticles (at −80° C.) were further dried.

A second set of BSA-loaded PLGA microparticles (at −178° C.) were prepared by printing the same ink into a plate containing liquid nitrogen (at −178° C.) by a 3D bioprinter (extrusion-based printing) with a printing speed of 50 mm/s, under a pressure of 200 kPa. The flash frozen BSA-loaded PLGA microparticles (at −178° C.) were further dried.

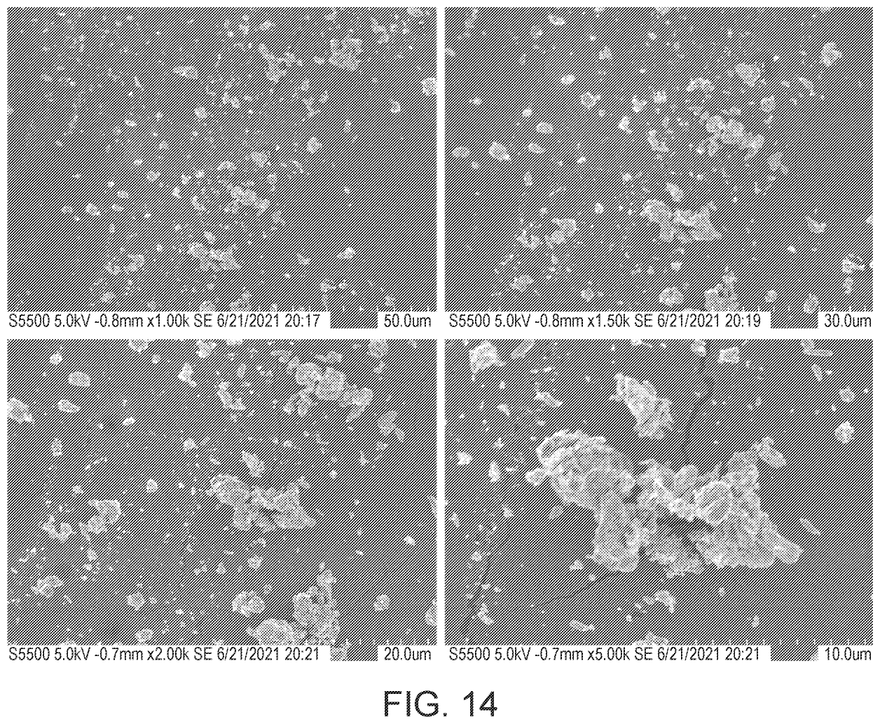

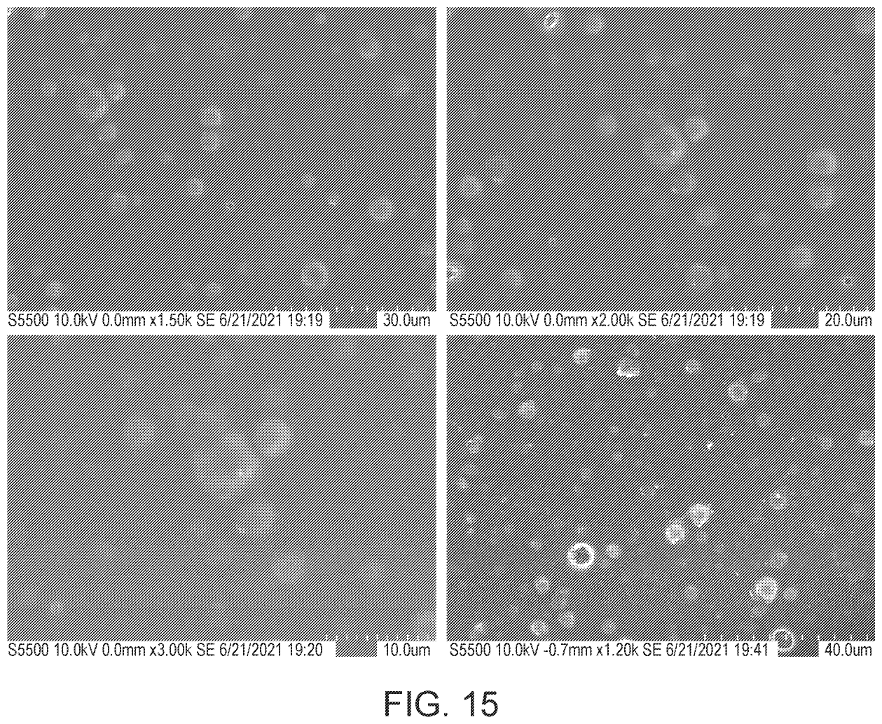

Samples of the BSA-loaded PLGA microparticles were subjected to imaging using scanning electron micrography (SEM). Images of BSA-loaded PLGA microparticles (at −80° C.) are shown in FIG. 14 and images of BSA-loaded PLGA microparticles (at −178° C.) are shown in FIG. 15.

TABLE 3
Formulation for BSA-loaded microparticles

| | | Amount | |
|---|---|---|---|
| Ingredient | Purpose | per batch | |
| BSA | active pharmaceutical | 3 | mg |
| | ingredient | | |
| PLGA | Polymer | 30 | mg |
| PVA | Stabilizer | 165 | mg |
| Chloroform | Organic solvent | 2 | ml |
| Sucrose | Cryoprotectant | 250 | mg |

### Example 5

PLGA microparticles may be encapsulated with ovalbumin (OVA) as a model protein therapeutic agent. Unless otherwise stated, the composition of each phase of the PLGA microparticles included the following inner aqueous phase, organic phase, and outer aqueous phase. The inner aqueous phase contained 15 mg/mL ovalbumin in 10 mM phosphate buffer saline (PBS, pH 7.4). The organic phase was a 20 mg/mL PLGA solution in dichloromethane (DCM). The outer aqueous phase consisted of 0.5% w/v PVA. A 25 gauge nozzle was used for extrusion.

FIG. 16 shows SEM images of the OVA-loaded PLGA microparticles prepared through different cycles of extrusions and printing pressure. As the results show, pressure and extrusion cycles may play a pivotal role for determination of particles size. Increasing pressure may result in particles size reduction. The same results may be obtained when increasing extrusion cycles (e.g., higher retention time at certain shear force resulted from the high pressure at a narrow channel (25 gauge nozzle)). SMART may also be capable of producing porous microparticles by adjusting the extruding pressure. This feature can be beneficial for immobilization of cells and enzymes in spherical polymer supports that require porous morphology for better access to their substrates as well as higher immobilization density.

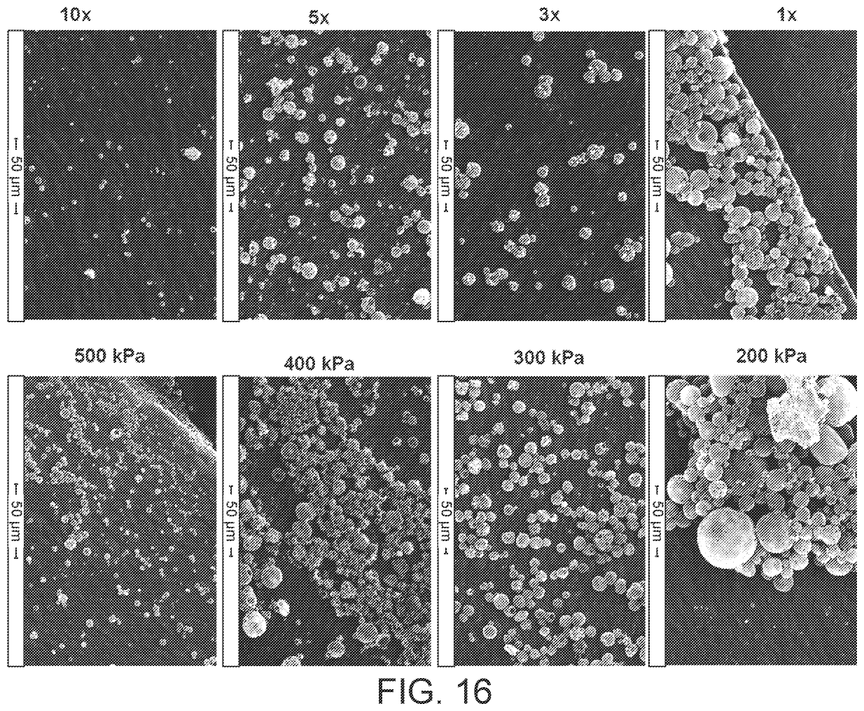

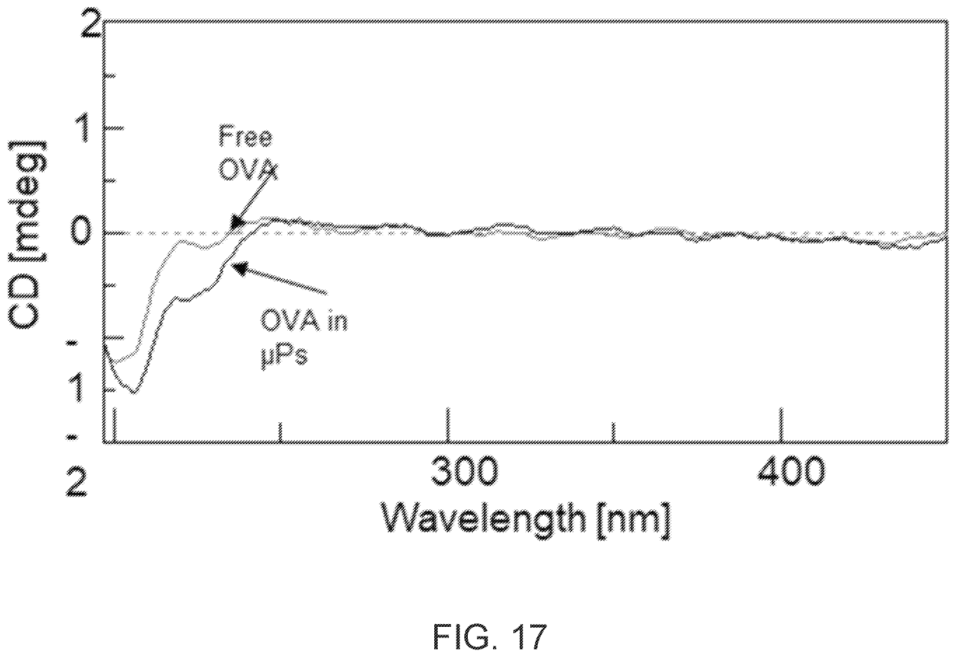

FIG. 17 shows a circular dichroism (CD) spectrum of free unprocessed ovalbumin and the encapsulated ovalbumin by SMART. Comparison of presented CD spectrums show that the secondary conformational structure of the model protein (OVA) has not changed after processing with the present technology. This proves that SMART may be compatible with proteins and polypeptide therapeutics, maintaining their secondary structure and, therefore, their biological activity.

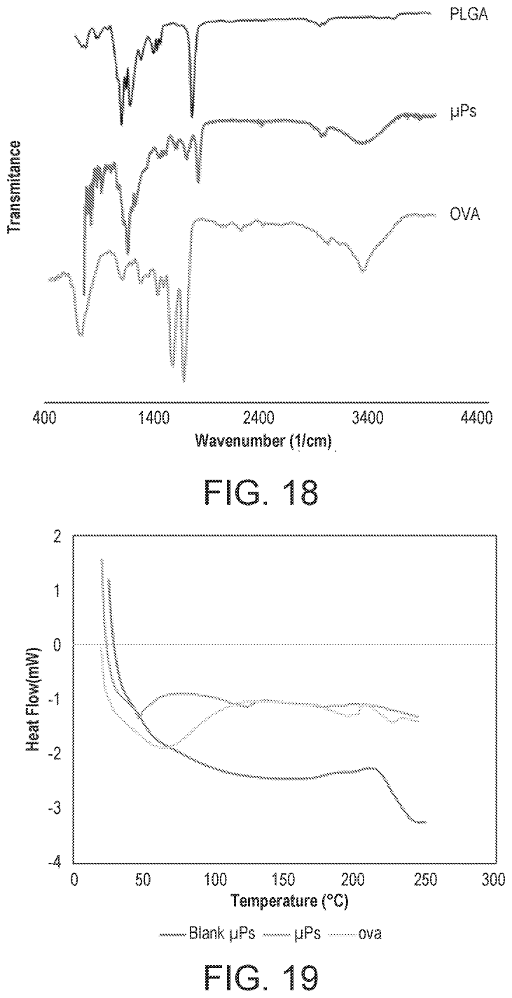

FIG. 18 shows Fourier-transform infrared (FTIR) spectrums of free unprocessed ovalbumin and the lyophilized OVA encapsulated PLGA microparticles. Comparison of presented spectrums confirms that the protein payload, OVA, has not undergone any chemical reaction and its chemical structures remains intact after the SMART process.

FIG. 19 shows differential scanning calorimetry of free unprocessed ovalbumin compared with OVA loaded PLGA microparticles and blank PLGA microparticles. The graphs show that the thermal property of the encapsulated protein remains unchanged.

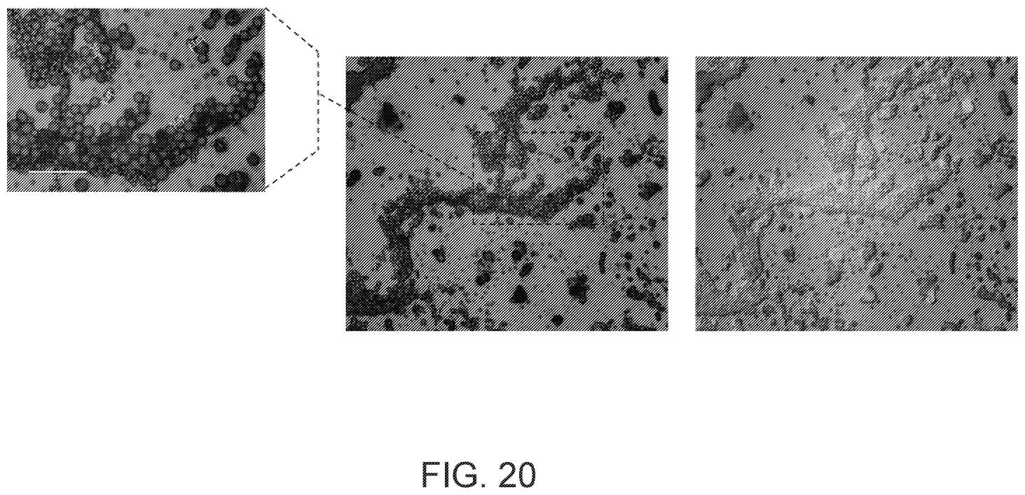

FIG. 20 shows fluorescent images of ovalbumin (OVA)-fluorescein isothiocyanate (FITC) loaded poly (lactide-co-glycolide) (PLGA) microparticles. Fluorescently labeled FITC-OVA was encapsulated into PLGA microparticles using SMART. The fluorescent microscope mapped the loading content of the prepared formulation. The results confirm that the protein payload is incorporated in the microparticles and can be carried by them as delivery vehicles.

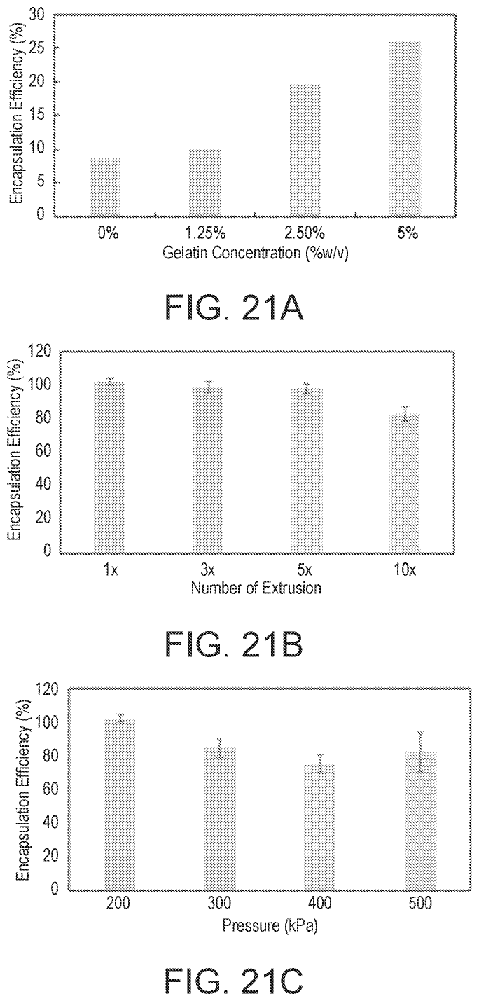

FIGS. 21A-21C shows the effect of (FIG. 21A) gelatin concentration, (FIG. 21B) number of extrusion cycles and (FIG. 21C) extrusion pressure on OVA encapsulation efficiency. Encapsulation efficiency may be defined as the percentage of loaded drug within the microparticles out of the total amount that was used. Results show the addition of an excipient to the inner aqueous phase where the protein exist may increase EE %. This observation may be due to the increasing of the viscosity of the inner aqueous upon addition of gelatin. Higher viscosity may decrease the diffusion rate of the protein out of the polymer matrix, which may result in entrapment of a higher amount of OVA within the microparticles. Increasing extrusion cycles to five times may not affect the EE % although it may have an effect on particles size. This shows SMART is capable of producing smaller particles without any reduction in their loading content. However, increasing the extrusion cycles to ten times may slightly reduce the loading. This may be associated with the limitations accompanied by that batch production such as loss of an amount of product during each cycle. The problem may be addressed in continuous SMART setup where the product loss may be much less due to usage of high volumes of the inks. Extrusion pressure may initially reduce the EE % due to facilitating the protein scape from the polymeric particles upon applying a force. However, this effect may reach its highest threshold at 300 kPa as no reduction is observed by increasing pressure from 300 kPa to 500 kPa.

Production yield may be calculated by Equation 1:

$$Yield ( \% ) = 100 \times \frac{Mass of lyophilized μ Ps}{Mass of used polymer + drug} \qquad ( 1 )$$

The yield was measured to be 78.3±9.4% for batch production of OVA-loaded PLGA microparticles. Therefore, SMART results in high production yield even by the low volume batch process.

### Example 6

Example 6 is directed to the production of chitosan (CS) microparticles incorporating ovalbumin as a model protein therapeutic agent. Low molecular weight CS was dissolved in 0.02% v/v acetic acid and used as the polymeric carrier. Drug solution contained 15 mg/mL ovalbumin in 10 mM phosphate buffer saline (PBS, pH 7.4). Sodium sulfate 10% w/v was used as a crosslinker to fix and stabilize CS microparticles. The three components were mixed together with a volume ratio of 4:1:1 (polymer:drug:crosslinker) and extruded at 600 kPa using a 25 gauge nozzle. Extrusion was followed by immediate flash freezing of the particles by liquid nitrogen and lyophilized. FTIR and CD spectrums of the powdered microparticles revealed that the protein payload remains intact after processing by SMART even without the presence of cryoprotectant.

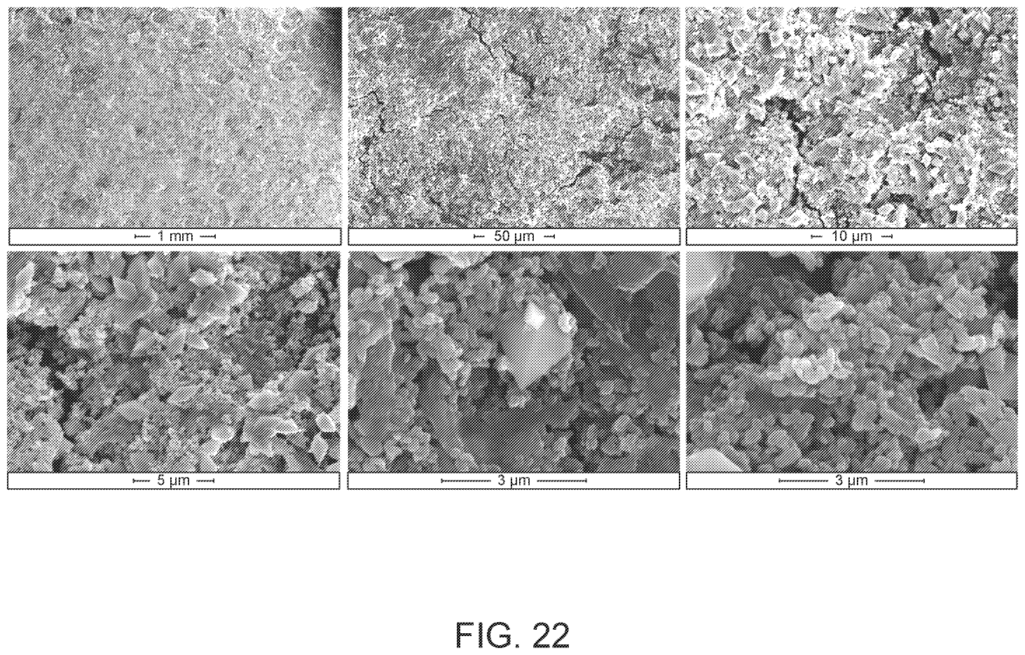

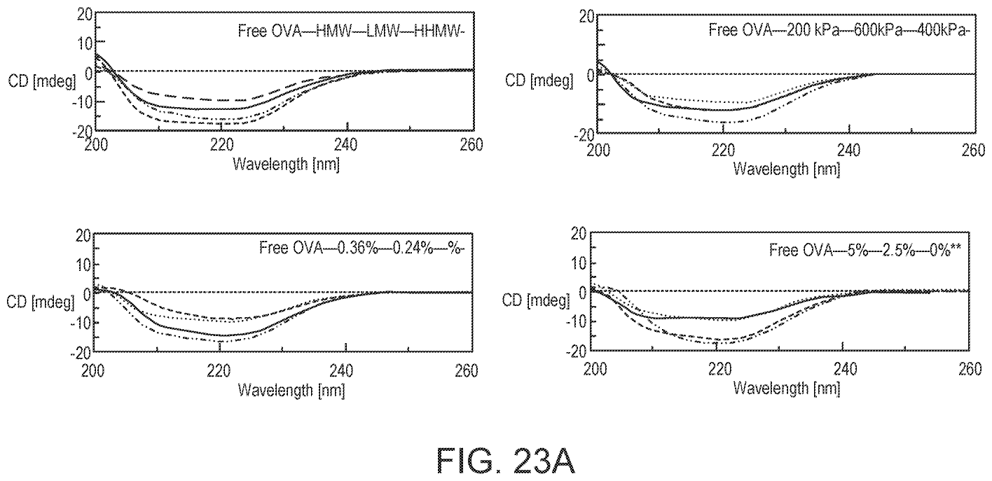

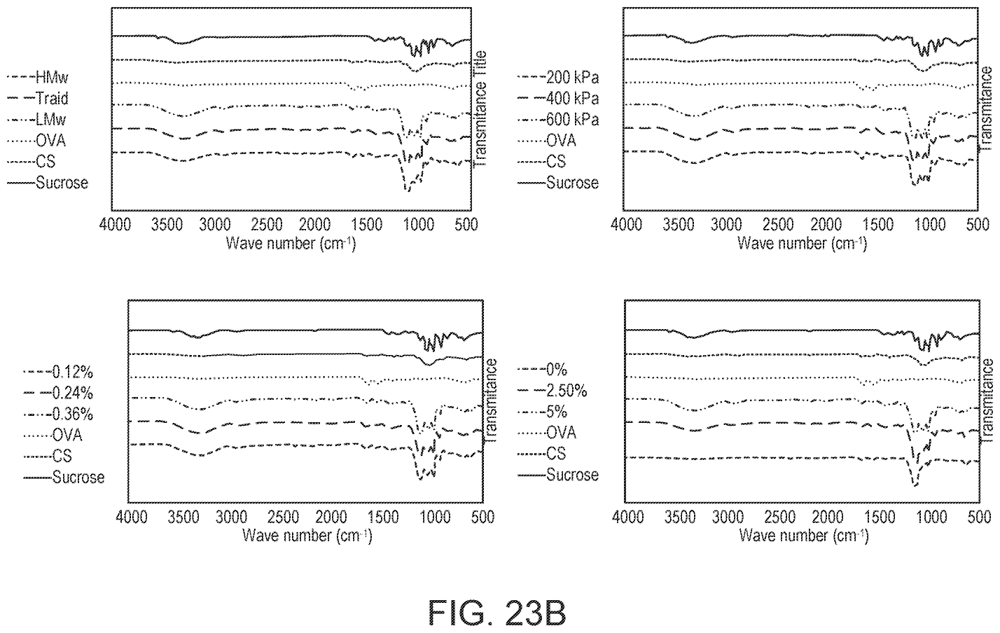

FIG. 22 shows scanning electron micrograph images of the ovalbumin (OVA) loaded chitosan (CS) microparticles at various magnifications. FIGS. 23A-23B shows characterization of ovalbumin (OVA) loaded chitosan (CS) microparticles made with various operational conditions. Specifically, FIG. 23A shows confirmation of payload integrity by CD and FIG. 23B shows the FTIR spectrum. In this study, effect of CS concentration, molecular weight of CS, extrusion pressure and concentration of a cryoprotectant (sucrose) was studied on structure of the protein. Results confirm that SMART technology with CS as a microcarrier is also compatible with the protein therapeutics.

### Example 7

Biodegradable microparticles have been extensively used as delivery vehicles for a variety of pharmaceutical dosage forms or drug delivery systems. Conventional microparticle formulation strategies include solvent displacement and emulsion evaporation technique. The present embodiments employed a first-in-class 3D printing concept to fabricate polymeric microparticle by a 3D printer. SMART combines extrusion-based printing with emulsion evaporation technique to fabricate a small molecule drug (e.g., 6-thioguanine (6-TG) loaded poly (lactide-co-glycolide) (PLGA) microparticle). Compared to conventional emulsion evaporation method, SMART employed the shear force exerted by the syringe nozzle rather than the sonication energy to generate micro-sized emulsion droplets. Furthermore, the shear force given by the 3D printer was controllable and consistent since the emulsion was extruded from the nozzle under a preset printing speed and pressure. The formulated SMART microparticle exhibited spherical structure with size distribution ˜1-3 μm in diameter and reached ˜100% drug release at 10 h. Also, the papain-like protease (PLpro) inhibition efficacy of 6-TG was maintained during the printing process under different printing parameters. To further predict the 6-TG drug loading efficiency (DLE) of SMART microparticles, comprehensive design of experiment (DoE) studies and machine learning modeling were performed with independent variables, including initial drug amount, printing speed, printing pressure, and nozzle size. It is interesting nearly all the machine learning models, especially decision tree (DT), demonstrated superior predictive performance compared to DoE regression models. Moreover, according to both DoE analysis and machine learning modeling, drug amount was the most influential formulation factor among the four process parameters. Overall, the present embodiments encompass the first case of using 3D printing concept to produce microparticle formulation with precise control over size of the resultant particles. Moreover, comprehensive DoE analysis and machine learning modeling were performed to predict the DLE. The predictive accuracy of different models were calculated and compared. The significance of process parameters was first evaluated by DoE statistical analysis, which were further validated based on feature importance ranking by machine learning modeling. Such systemic approach demonstrates great promises in optimizing microparticle formulations with desired properties, identifying critical formulation factors, and streamlining the development of microparticles with programmable pharmaceutical attributes, representing a new paradigm for digital pharmaceutical science.

PLGA (50:50 lactic-glycolic ratio) was obtained from Ashland. 6-TG was purchased from Chem-Implex Inc. (Wood Dale, IL, USA). SARS-CoV2-PLpro was purchased from Cayman Chemicals (Ann Arbor, MI, USA). Dithiothreitol (DTT) and chloroform were purchased from Thermo Fisher Scientific. Z-Arg-Leu-Arg-Gly-Gly-AMC (Z-RLRGG-AMC) acetate salt was provided from Bachem Americas Inc. (Torrance, CA, USA). Poly(vinyl alcohol) (PVA, Average Mw 30,000-70,000) was obtained from Sigma-Aldrich.

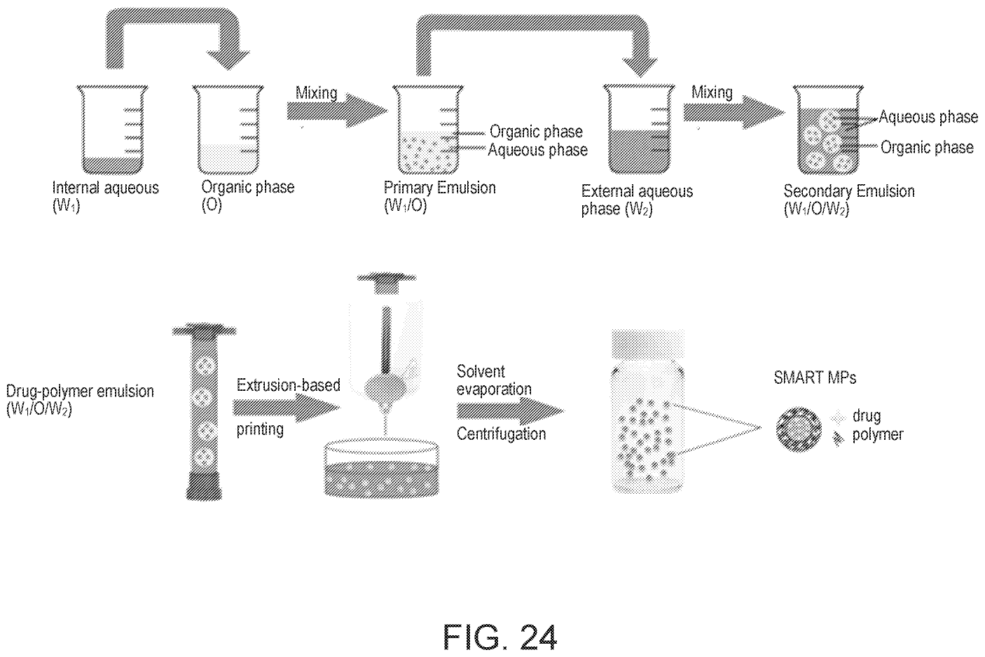

As shown in FIG. 24, 6-TG loaded SMART microparticles were prepared using the combination of water-in-oil-in-water (water/oil/water) double emulsion solvent evaporation and extrusion-based bioprinting technique. Briefly, 6-TG (6 mg or 9 mg) was dissolved in 0.11M NaOH PVA solution (1% w/v) to generate the internal aqueous phase. The inner aqueous phase was further added to organic phase and PLGA was dissolved in chloroform (7.5 mg/ml), followed by complete mixing to form primary emulsion. The primary emulsion was further added to the external aqueous phase, 2% (w/v) PVA solution, followed by complete mixing to generate the secondary emulsion. The secondary emulsion was then transferred into a syringe for the Cellink BioX bioprinter and processed through extrusion-based printing under different printing parameters, including nozzle size (20 or 25 gauge), printing speed (10 or 20 mm/s), and printing pressure (100 or 200 kPa). After the evaporation of organic solvent, microparticles were centrifuged to remove residual PVA and unencapsulated 6-TG. The washed SMART microparticles were then collected and characterized.

The morphology and size of SMART microparticles were examined by scanning electron microscopy (SEM) (Hitachi S-5500 SEM/STEM, Hitachi, USA). 10 μl microparticle suspension was deposited on the aluminum foil and dried overnight at room temperature. The dried samples were subsequently coated with gold by vacuum sputtering (EMS Sputter Coater, Hatfield, USA) prior to imaging.

The DLE of SMART microparticles was determined by measuring the weight of total encapsulated drug divided by the total weight of microparticles. The amount of 6-TG encapsulated into the SMART microparticles was quantified using UV-Visible absorbance analysis. Briefly, prepared SMART microparticles were dispersed in 0.1M NaOH at a concentration of 1 mg/ml and incubated overnight in room temperature. The dispersion was centrifuged and 100 μl of the supernatant was collected for UV analysis. The absorbance value was measured at 322 nm using the Infinite M200 Plate Reader (Tecan, NC, USA). The DLE was estimated by Equation 2:

$$DLE ( \frac{μ g}{mg} ) = \frac{Weight of drug encapsulated within MPs}{Weight of MPs} \qquad ( 2 )$$

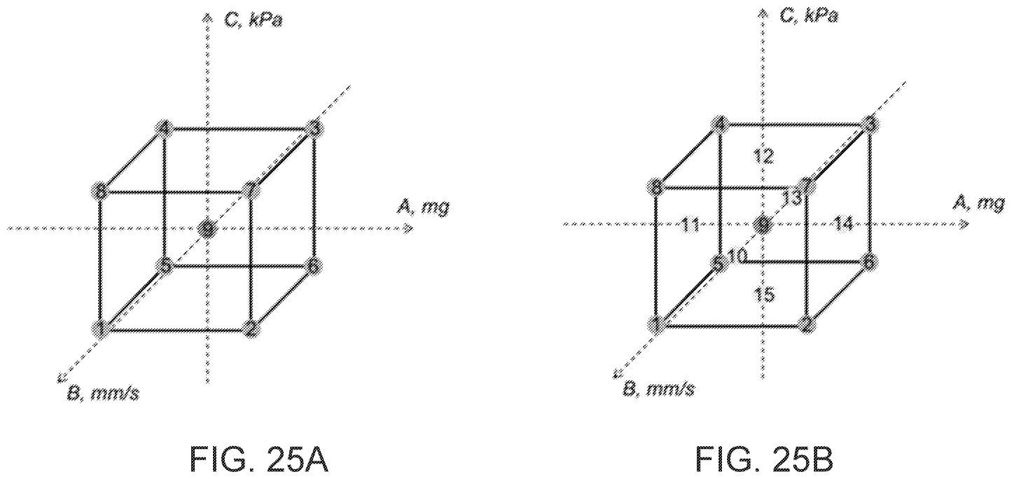

Minitab software was used for the DoE analysis. Four-factor/two-level full factorial design was initially used to explore the relationship between process parameters and DLE as shown in Table 4. In the present study, initial drug amount (A, mg), printing speed (B, mm/s), printing pressure (C, kPa), and nozzle size (D, gauge) were identified as independent variables and DLE was selected as the response variable. Stepwise selection was employed to remove statistically non-significant terms from the model from a higher order to a lower order to obtain the regression equation. To examine the curvature effect of the fitted data, center points were introduced into the factorial design. Since nozzle size is considered as a discrete variable, it was held constant at 20 gauge due to the production of a higher DLE. Next, three-factor/two-level full factorial design with center point was used to investigate whether there is nonlinear relationship between the DLE and process parameters as shown in Table 5. Due to the presence of curvature effect, face-centered CCD was used to include squared terms into the regression model as shown in Table 6. In addition to center points and factorial points, CCD was further augmented with a group of axial points to allow the estimation of nonlinear relationship between the DLE and process parameters. Similarly, statistically non-significant terms were removed from the model step by step from a higher order to a lower order to obtain the final CCD regression equation. The schematic representation of three-factor/two-level factorial design and face-center CCD are shown in FIGS. 25A-25B.

TABLE 4

| | | Drug | Printing | Printing | Nozzle |
|---|---|---|---|---|---|
| | Factor | amount | speed | Pressure | size |
| Trial | levels | (mg) | (mm/s) | (kPa) | (gauge) |
| 1 | 1, −1, 1, 1 | 9 | 10 | 200 | 25 |
| 2 | −1, 1, −1, −1 | 6 | 20 | 100 | 20 |
| 3 | 1, 1, −1, −1 | 9 | 20 | 100 | 20 |
| 4 | −1, 1, 1, 1 | 6 | 20 | 200 | 25 |
| 5 | 1, −1, 1, −1 | 9 | 10 | 200 | 20 |
| 6 | 1, −1, −1, 1 | 9 | 10 | 100 | 25 |
| 7 | −1, 1, −1, 1 | 6 | 20 | 100 | 25 |
| 8 | −1, −1, −1, 1 | 6 | 10 | 100 | 25 |
| 9 | −1, −1, 1, −1 | 6 | 10 | 200 | 20 |
| 10 | 1, 1, −1, 1 | 9 | 20 | 100 | 25 |
| 11 | −1, −1, −1, −1 | 6 | 10 | 100 | 20 |
| 12 | 1, −1, −1, −1 | 9 | 10 | 100 | 20 |
| 13 | 1, 1, 1, 1 | 9 | 20 | 200 | 25 |
| 14 | 1, 1, 1, −1 | 9 | 20 | 200 | 20 |
| 15 | −1, 1, 1, −1 | 6 | 20 | 200 | 20 |
| 16 | −1, −1, 1, 1 | 6 | 10 | 200 | 25 |

TABLE 5

| | | Drug | Printing | Printing |
|---|---|---|---|---|
| | Factor | amount | speed | pressure |
| Trial | levels | (mg) | (mm/s) | (kPa) |
| 1 | −1, 1, −1 | 6 | 20 | 100 |
| 2 | 1, 1, −1 | 9 | 20 | 100 |
| 3 | 1, −1, 1 | 9 | 10 | 200 |
| 4 | −1, −1, 1 | 6 | 10 | 200 |
| 5 | −1, −1, −1 | 6 | 10 | 100 |
| 6 | 1, −1, −1 | 9 | 10 | 100 |
| 7 | 1, 1, 1 | 9 | 20 | 200 |
| 8 | −1, 1, 1 | 6 | 20 | 200 |
| 9 | 0, 0, 0 | 7.5 | 15 | 150 |

TABLE 6

| | | Drug | Printing | Printing |
|---|---|---|---|---|
| | Factor | amount | speed | pressure |
| Trial | levels | (mg) | (mm/s) | (kPa) |
| 1 | −1, 1, −1 | 6 | 20 | 100 |
| 2 | 1, 1, −1 | 9 | 20 | 100 |
| 3 | 1, −1, 1 | 9 | 10 | 200 |
| 4 | −1, −1, 1 | 6 | 10 | 200 |
| 5 | −1, −1, −1 | 6 | 10 | 100 |
| 6 | 1, −1, −1 | 9 | 10 | 100 |
| 7 | 1, 1, 1 | 9 | 20 | 200 |
| 8 | −1, 1, 1 | 6 | 20 | 200 |
| 9 | 0, 0, 0 | 7.5 | 15 | 150 |
| 10 | 0, 1, 0 | 7.5 | 20 | 150 |
| 11 | −1, 0, 0 | 6 | 15 | 150 |
| 12 | 0, 0, 1 | 7.5 | 15 | 200 |
| 13 | 0, −1, 0 | 7.5 | 10 | 150 |
| 14 | 1, 0, 0 | 9 | 15 | 150 |
| 15 | 0, 0, −1 | 7.5 | 15 | 100 |

In vitro release of 6-TG from SMART microparticles was determined using a previously reported dialysis technique. Microparticles (4 mg/mi) were dispersed in release buffer (PBS, pH 7.4) and transferred to the dialysis tubes (SnakeSkin Dialysis Tubing, 3.5K MWCO, Life Technologies Corpora, CA, USA). Dialysis tubes were immersed in 10 ml release buffer contained in a 20 ml glass vial. Sample vials were incubated in the shaker at 37° C. at 200 RPM. At pre-determined time points, 0.5 ml sample was collected from each vial, which was then refilled with fresh release buffer. The amount of 6-TG from collected samples was quantified by UV analysis as described above. Each experiment was performed in triplicate.

For in vitro SARS-CoV PLpro inhibition assay, the SARS-Cov PLpro and fluorogenic peptide Z-RLRGG-AMC were used as the enzyme and substrate, respectively. The hydrolysis of the AMC-peptide bond by SARS-CoV PLpro greatly increased the fluorescence of AMC moiety, allowing the enzyme activity to be accurately determined. Reactions were performed in a total volume of 200 μl 20 mM Tris-buffer (pH 8.0), including the following components: 4 mM DTT, M Z-RLRGG-AMC, 60 nM SARS-Cov PLpro, 2% dimethyl sulfoxide (DMSO), and varying concentrations of free 6-TG and 6-TG loaded SMART microparticles (0-200 μM). The amount of SMART microparticles was based on the DLE to obtain the same concentration as free 6-TG. The fluorescence intensity was measured at an excitation wavelength of 360 nm and an emission wavelength of 460 nm using the Synergy H1 Multi-Mode Plate Reader (BioTek instruments inc, USA).

The machine learning models were trained on the DLE dataset, which was split into training subset (85%) and test subset (15%). The training subset was used for model construction and tuning model hyper-parameters, while the test subset was used to evaluate the model prediction accuracy on unknown data. Since the present dataset is not very large, ten-fold cross validation method was used for modeling and tuning the model hyper-parameters. For the ten-fold cross validation strategy, the training dataset was split into ten subsets. Nine subsets were used to train the machine learning models while the last subset was used to validate the models. Finally, the test dataset was used to evaluate the performance of optimized machine learning models.

Five machine learning algorithms were applied to construct regression models including DT, RF, KNN, LightGBM, and XGBoost. The LightGBM and XGBoost models were constructed using the LightGBM and XGBoost package in Python, respectively. Other models were constructed using the scikit-learn package in Python. For DT, the maximum depth of the tree and the minimum number of samples required to split an internal node were set to 4 and 4, respectively. For RF, the number of trees, the maximum tree depth, and the minimum number of samples required to split an internal node were set to 50, 4, and 2, respectively. For KNN, the number of neighbors was set to 2. For XGBoost, the boosting learning rate, number of gradient boosted trees, gamma, maximum tree depth, minimum sum of instance weight needed in a child, subsample ratio, and subsample ratio of columns were set to 0.2, 50, 0.4, 3, 6, 0.9, and 0.8, respectively. For LightGBM, the boosting learning rate, number of gradient boosted trees, gamma, maximum tree depth, minimum sum of instance weight needed in a child, subsample ratio, and subsample ratio of columns were set to 0.1, 100, 2, 3, 0.5, 0.5, respectively.

In machine learning modeling, statistical measures including coefficient of determination (R²), mean absolute error (MAE), and root mean square error (RMSE) are widely adopted to evaluate the model performance for regression tasks. In this study, R², MAE, and RMSE were selected to evaluate the model predictive accuracy for DLE. In statistics, R² is the proportion of total variation in the response variable that can be explained by the independent variable(s). R² is calculated by Equation 3:

$${R}^{2} - \frac{{\sum }_{i = 1}^{n} {( {x}_{i} - \overline{y} )}^{2}}{{\sum }_{i = 1}^{n} {( {y}_{i} - \overline{y} )}^{2}} \qquad ( 3 )$$

In the previous equation, yᵢ=actual value, xᵢ=predicted value, y̅=the mean of actual values, and n=total number of samples. MAE refers to the average absolute difference between the predicted value and the true value of an observation. MAE is calculated by Equation 4:

$$MAE = \frac{{\sum }_{i = 1}^{n} \vert {y}_{i} - {x}_{i} \vert}{n} \qquad ( 4 )$$

RMSE is the standard deviation of the residuals in the regression analysis. A residual is the error between the predicted value and the observed actual value. RMSE is calculated by Equation (5):

$$RMSE = \sqrt{\frac{{\sum }_{i = 1}^{n} {( {y}_{i} - {x}_{i} )}^{2}}{n}} \qquad ( 5 )$$

To further validate the predictive accuracy and the generalization of different DoE regression models and machine learning models, the experimental DLE of newly developed SMART microparticles that were not listed in the above DoE datasets were compared with the predicted DLE calculated by different DoE regression models and machine learning algorithms. In the validation dataset, the process parameters (drug amount, printing speed, printing pressure, and nozzle size) and DLE were selected as the independent variables and prediction target, respectively. The experimental data with nozzle size at 20 gauge were used to validate the face-centered CCD model. The process parameter values of newly prepared SMART microparticles for model validation were listed in Table 7.

TABLE 7

| | Drug | Printing | Printing | Nozzle |
|---|---|---|---|---|
| | amount | speed | pressure | size |
| Trial | (μg/mg/) | (mm/s) | (kPa) | (gauge) |
| 1 | 8 | 10 | 105 | 20 |
| 2 | 6.5 | 17 | 140 | 25 |
| 3 | 7 | 19 | 120 | 20 |
| 4 | 8 | 12 | 153 | 25 |
| 5 | 6.5 | 14 | 179 | 25 |
| 6 | 6.5 | 15 | 170 | 25 |
| 7 | 8 | 14 | 148 | 20 |
| 8 | 8.5 | 12 | 127 | 25 |
| 9 | 6.5 | 10 | 115 | 20 |
| 10 | 8.5 | 15 | 158 | 20 |
| 11 | 6.5 | 13 | 110 | 20 |
| 12 | 6.5 | 19 | 130 | 20 |
| 13 | 8 | 18 | 183 | 20 |
| 14 | 7 | 18 | 150 | 20 |
| 15 | 8 | 13 | 182 | 25 |
| 16 | 8.5 | 17 | 168 | 25 |
| 17 | 8 | 15 | 194 | 25 |
| 18 | 8.5 | 16 | 135 | 20 |
| 19 | 7 | 12 | 180 | 20 |
| 20 | 6.5 | 16 | 170 | 20 |

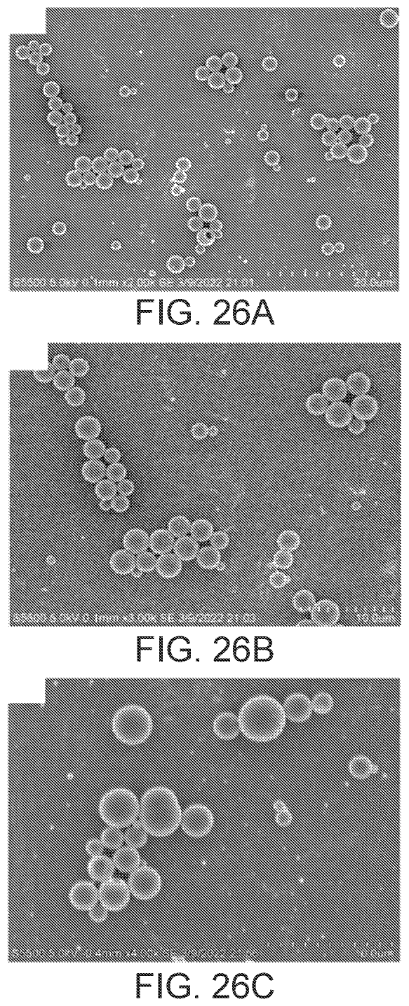

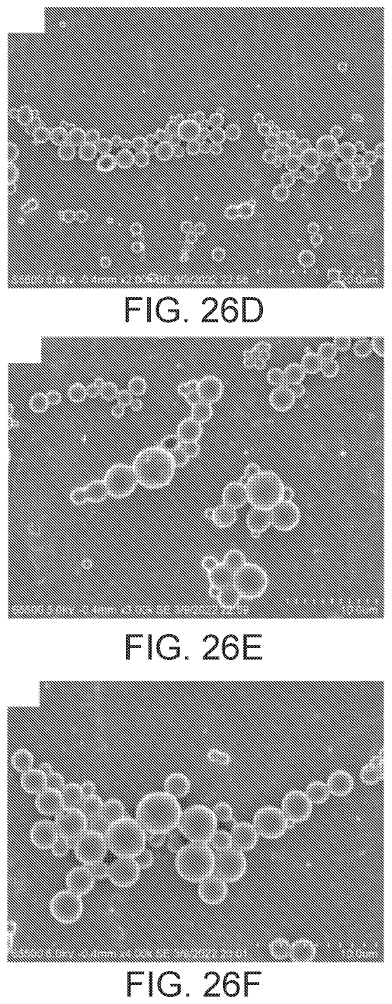

SMART technology was utilized to formulate polymeric drug-loaded microparticles. FIGS. 26A-26F show SEM images of 6-TG loaded SMART microparticles fabricated under different printing parameters. FIGS. 26A-26C show a printing speed of 15 mm/s, a printing pressure of 200 kPa, and a nozzle size of 20 gauge. FIGS. 26D-26F show a printing speed of 20 mm/s, a printing pressure of 150 kPa, and a nozzle size of 20 gauge. As shown, SMART microparticles exhibited spherical structure with smooth surface. The particle size distribution was around 1-3 μm in diameter.

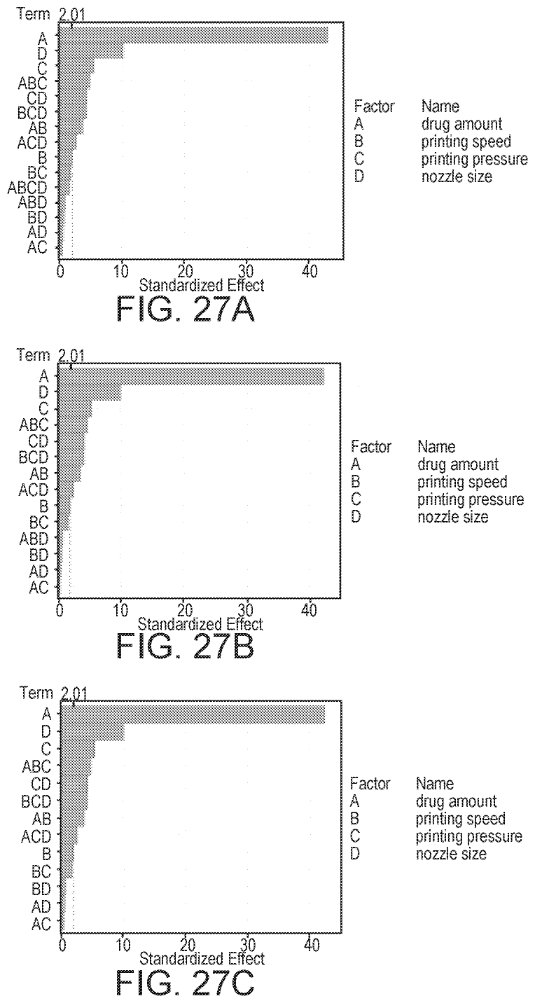

Four-factor/two-level full factorial design (n=4) was initially used to investigate the relationship between process parameters (drug amount, printing speed, printing pressure, and nozzle size) and DLE. The Pareto chart with a reference line is employed to examine which factors and their interactions are statistically significant. Term bars that cross the reference line (α=0.05) are statistically significant, whereas bars below the reference line are considered as statistically not significant. The longer the bars are, the more statistically significant the terms are, and the smaller P-values they have. As shown in FIG. 27A, the main effects of factors (A, B, C, D), 2-way interaction effects (A*B, A*C, A*D, B*C, B*D, C*D), 3-way interaction effects (A*B*C, A*B*D, A*C*D, B*C*D), and 4-way interaction effect (A*B*C*D) were all included in the Pareto chart. Next, the stepwise selection was employed to remove statistically non-significant terms from the model from a higher order to a lower order and started from 4-way interaction term A*B*C*D. Since the P-value of A*B*C*D is 0.122, higher than the significance level (α=0.05), A*BI*C*D term was first removed from the regression model and the Pareto chart without 4-way interaction term was obtained as shown in FIG. 27B. Among the four 3-factor interaction terms (A*B*C, A*B*D, A*C*D, B*C*D), only A*B*D term bar was below the reference line with P-value (0.386)>0.05, indicating the drug amount*printing speed*nozzle size interaction effect is not significant. The A*B*D term was then removed from the model. As is shown in FIG. 27C, bars of the remaining three 3-way interaction terms (A*B*C, A*C*D, B*C*D) were all below the reference line. Correspondingly, their P-values were all less than the significance level, indicating that A*B*C, A*C*D, and B*C*D significantly affected the response variable. Thus, all of them were kept within the current model. Similarly, all the lower order terms that comprise these higher order terms also need to be retained. Consequently, even though some 2-way interaction terms including B*C, B*D, A*D, and A*C, possess P-values higher than 0.05, none of them should be removed from the current regression model. The final polynomial regression model was expressed by equation 6 with R²=0.97. Table 8 shows the change of P-values during the stepwise removement of statistically non-significant terms, corresponding to the pareto charts in FIGS. 27A-27C.

$$DLE = - 79.1 + 14.97 * A + 4.103 * B + 0.2786 * C + 14.11 * D - 0.5673 * A * B - 0.04391 * A * C - 1.326 * A * D - 0.02096 * B * C - 0.639 * B * D - 0.1014 * C * D + 0.0 \; 03017 * A * B * C + 0.00818 * A * C * D + 0.004024 * B * C * D \qquad ( 6 )$$

TABLE 8

| | | P- | |
|---|---|---|---|
| | Initial P- | value (without | |
| | value (all | 4- | Final |
| | terms | way interaction | P- |
| Terms | included) | term) | value |
| Constant | <0.001 | <0.001 | <0.001 |
| A | <0.001 | <0.001 | <0.001 |
| B | 0.041 | 0.044 | 0.043 |
| C | <0.001 | <0.001 | <0.001 |
| D | <0.001 | <0.001 | <0.001 |
| A*B | 0.001 | 0.001 | 0.001 |
| A*C | 0.667 | 0.672 | 0.671 |
| A*D | 0.526 | 0.533 | 0.532 |
| B*C | 0.079 | 0.084 | 0.083 |
| B*D | 0.451 | 0.458 | 0.457 |
| C*D | <0.001 | <0.001 | <0.001 |
| A*B*C | <0.001 | <0.001 | <0.001 |
| A*B*D | 0.379 | 0.386 | — |
| A*C*D | 0.011 | 0.012 | 0.012 |
| B*C*D | <0.001 | <0.001 | <0.001 |
| A*B*C*D | 0.112 | — | — |

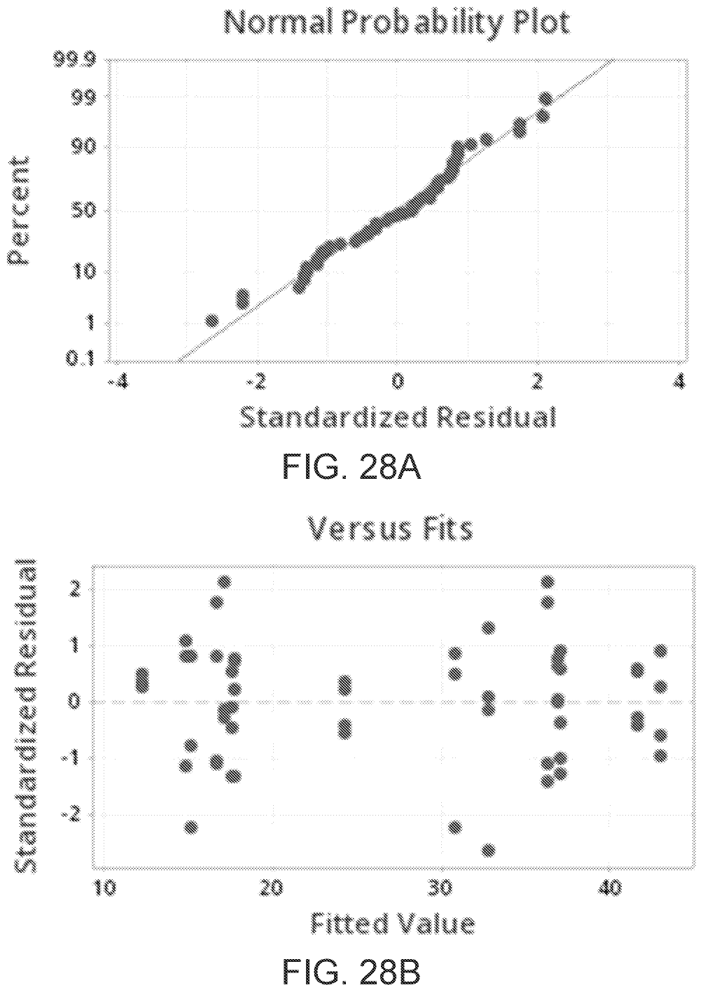

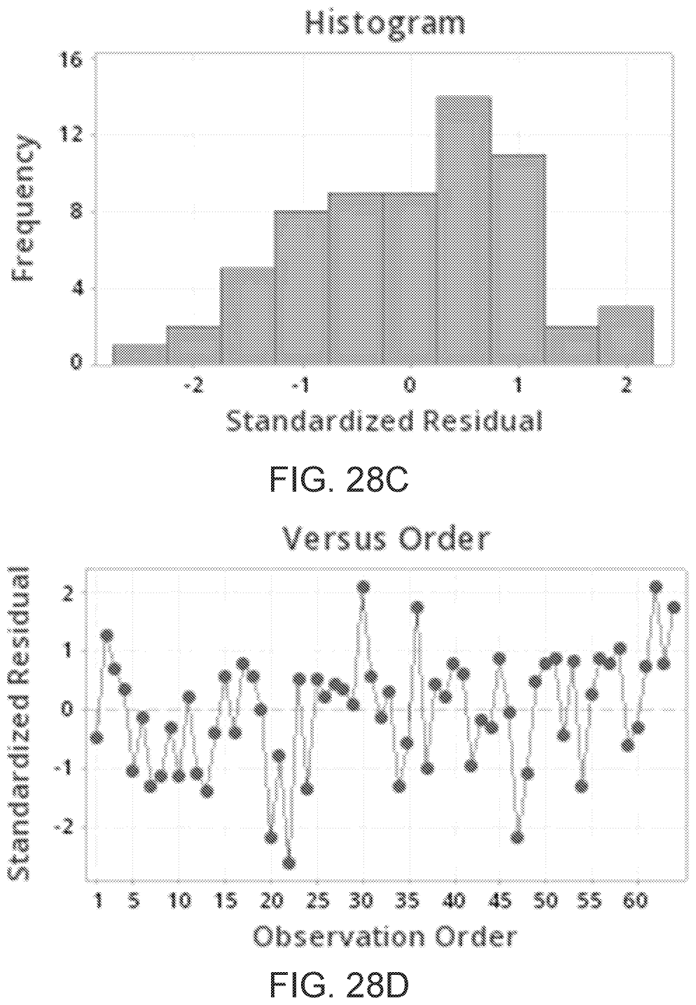

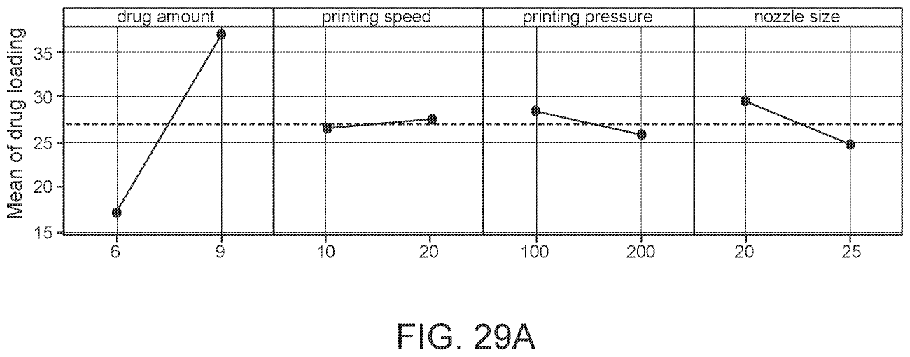

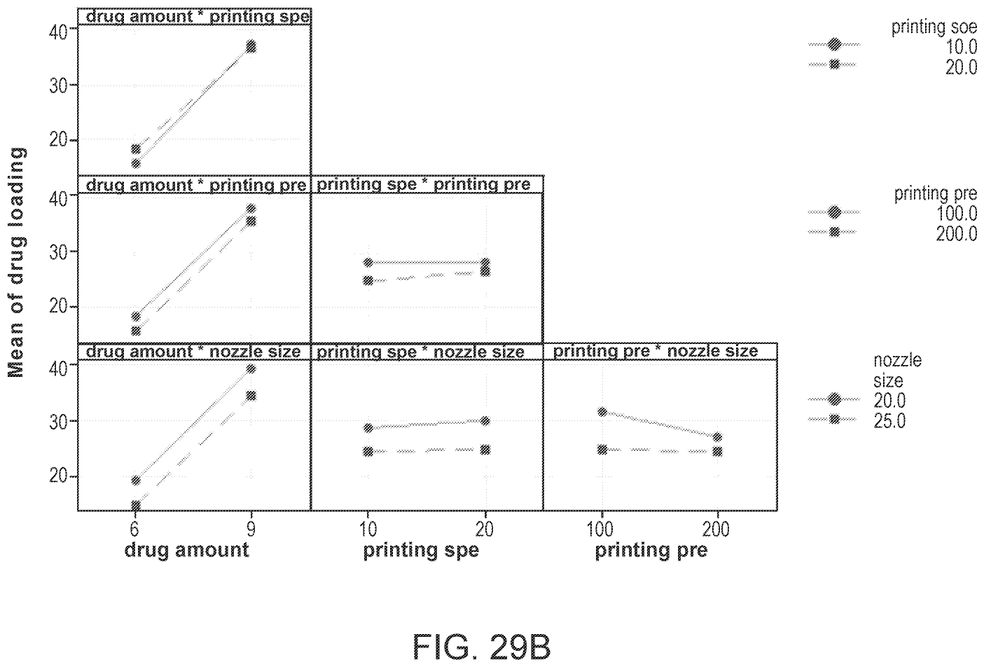

Residual plots are generally used to examine whether the ordinary least squares assumptions are satisfied for the current dataset. If these assumptions are met, the least square regression analysis would generate unbiased coefficient estimates with minimal variance. The normal probability plot is used to validate the assumption that the residuals are normally distributed. As shown in FIG. 28A, most data points clustered around the diagonal line, although a few points drifted away from the line at the end, indicating that the residuals could be considered as normally distributed. The residuals versus fits plot is used examine whether the residuals have a constant variance. As shown in FIG. 28B, the variance of residuals did not significantly change under different fitted values, suggesting that the equal variance assumption was satisfied. Furthermore, residual histogram is employed to assess whether the residuals are normal distributed. It can be observed from FIG. 28C that the residual distribution was very close to normal distribution. Moreover, the residuals versus order plot is utilized to verify the assumption that the residuals are not correlated with each other. As shown in FIG. 28D, residuals fell randomly around the centerline when displayed in time order. Also, no obvious pattern was exhibited under different observation orders and the residuals were all within three standard deviations, indicating that residuals were independent with each other and there were no outliers. Factorial plots are mainly used to illustrate the relationship between factors and response variables. Main effects plots are utilized to exhibit the relationship between response variable and individual factors. As shown in FIG. 29A, the main effect plot for drug amount displayed the greatest slope compared to the other three factors, implying that initial drug input significantly influenced the final DLE. The increase of initial drug amount could greatly improve final DLE. In contrast, the main effect plot for printing speed showed the lowest slope, indicating that printing speed did not affect the final DLE as much as the other three factors. Correspondingly, the P-value of printing speed was 0.043, slightly less than 0.05, indicating the printing speed was a marginally significant independent variable. In addition, the interaction plot is utilized to show how the relationship between response variable and one factor depends on the value of the second factor. In an interaction plot, parallel lines suggest that there is no interaction effect, whereas different slopes indicate the existence of interaction effect. As is shown in FIG. 29B, nonparallel lines with different slopes were observed in the drug amount*printing speed and printing pressure*nozzle size interaction plots, implying the presence of interaction between drug amount and printing pressure as well as between printing pressure and nozzle size. This can be confirmed by the P-values of A*B and C*D terms, which were much less than 0.01 as shown in Table 8. In contrast, parallel lines were observed in the middle four interaction plots, suggesting there was no interaction effect between the two factors in each plot. Accordingly, P-values of A*C, A*D, B*C, and B*D were much greater than the significance level.

The four-factor/two-level full factorial design without center point was able to analyze the factor main effects and their interaction effects. To further evaluate the curvature effect of the regression model and to investigate whether there is nonlinear relationship between the response variable and factors, center points were introduced into the factorial design. Center points are experiments where numeric continuous variables are set midway between their low and high levels. Since nozzle size is a discrete variable and previous results showed 20 gauge produced higher DLE than 25 gauge, it was held constant at 20 gauge for the following study. Herein, the values of center points (n=4) were set as 7.5 mg (drug amount), 15 mm/s (printing speed), and 150 kPa (printing pressure). As shown in Table 9, the P-value of Ct Pt term was significantly low (<0.001), indicating that the regression model had curvature effect and there was nonlinear relationship between the DLE and independent variables. Next, statistically non-significant terms were removed from the model step by step from a higher order to a lower order and started from 3-way interaction term A*B*C (P-value=0.091). The B*C and A*B*C terms possessed P-values greater than the significance level and thus were removed from the model. The P-value of the Ct Pt term in the final model was significantly less than 0.01, indicating that more advanced design is needed to explore the curvature effect of the model. Additionally, squared terms should be incorporated into the model to determine the optimal settings for each factor.

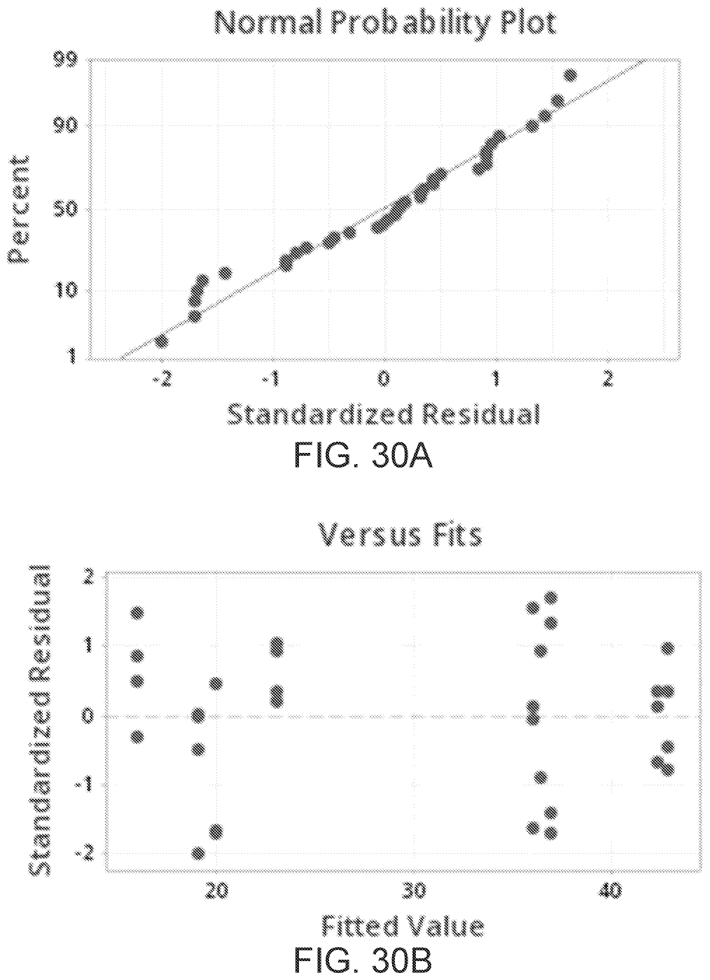

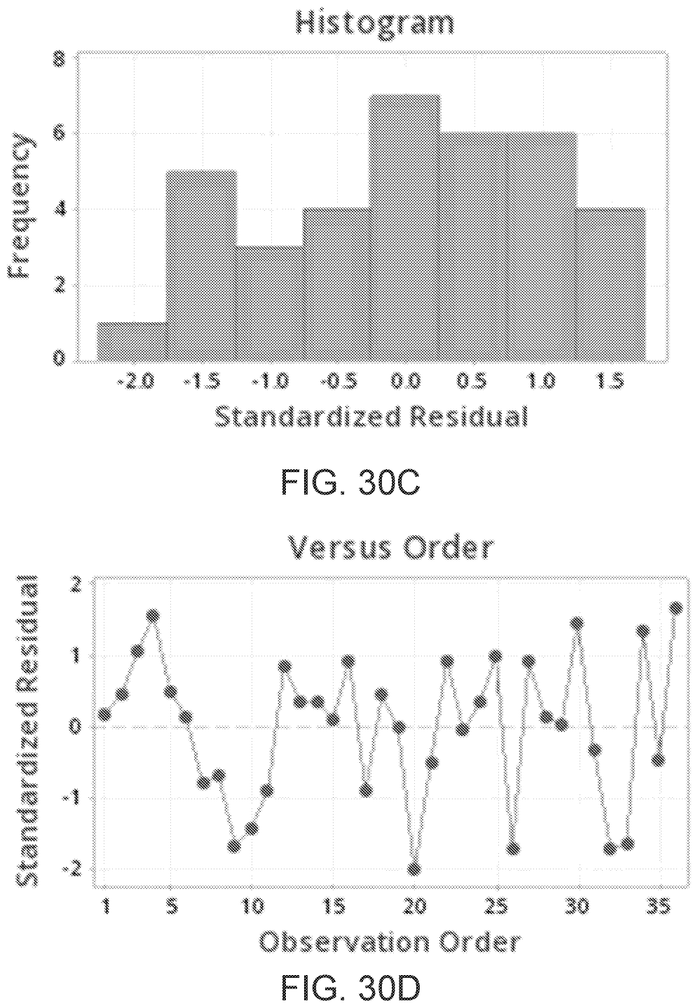

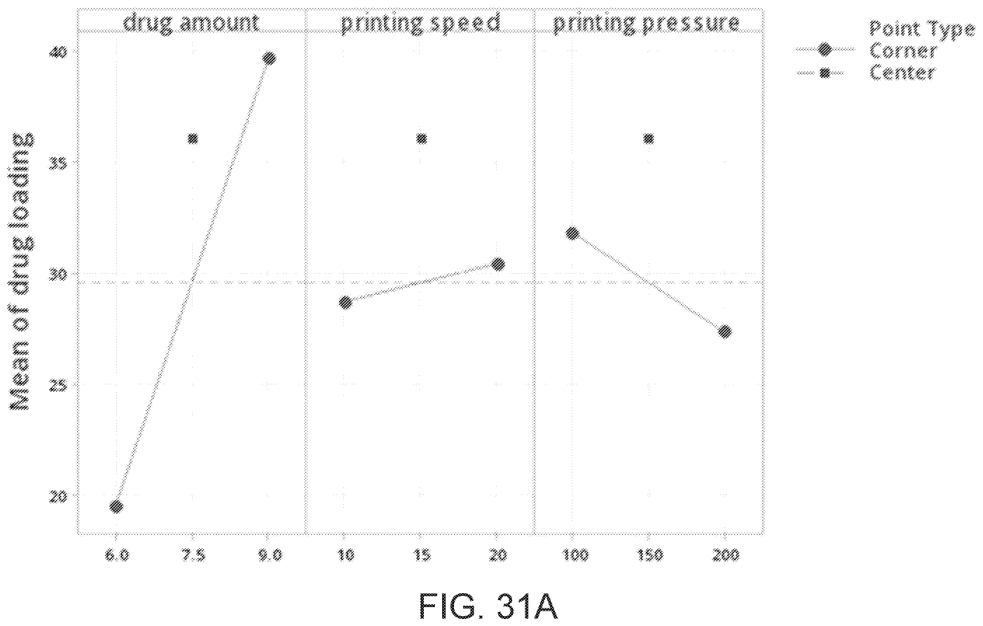

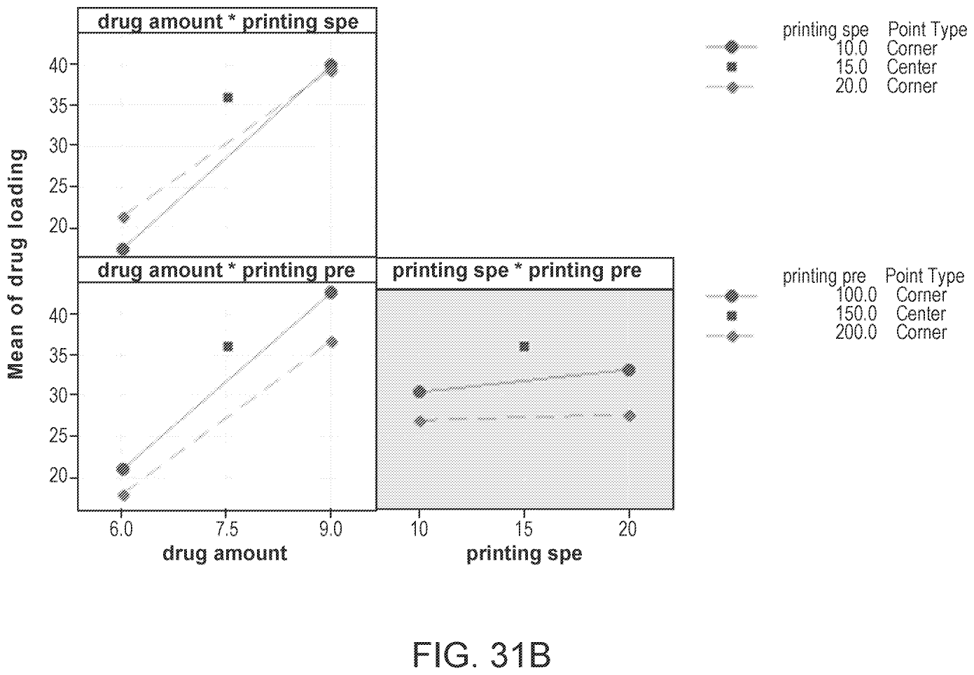

For three-factor/two-level full factorial design with the inclusion of center points (n=4), residual plots were created to examine whether the ordinary least squares assumptions are met for the current dataset. As shown in FIG. 30A, most data points clustered around the diagonal line, implying that the residuals followed normal distribution, which could be confirmed by the residual histogram shown in FIG. 30C. Furthermore, it was observed from FIG. 30B that the variance of residuals did not significantly change under different fitted values, indicating that the equal variance assumption was satisfied. Additionally, residuals randomly distributed around the centerline in the residuals versus order plot shown in FIG. 30D and no residual exceeded three standard deviations, indicating that residuals were uncorrelated with each other and there were no outliers. Moreover, factorial plots were generated to display the relationship between factors and response variable. The inclusion of center points helped to detect the curvature effect of the fitted data. It was shown that the response of center point did not lie in the average response of all the corner points in both main effects plot shown in FIG. 31A and interaction plot shown in FIG. 31B, indicating the presence of curvature effect of the current data. Furthermore, as shown in FIG. 31A, drug amount exhibited the greatest slope among the three factors, confirming the previous result that the initial drug input greatly affected the final DLE. On top of that, nonparallel lines occurred in drug amount*printing speed and drug amount*printing pressure interaction plots, indicating the existence of interaction between drug amount and printing speed or printing pressure.

TABLE 9

| | Initial P- | |
|---|---|---|
| | value (all | Final |
| | terms | P- |
| Term | included) | value |
| Constant | <0.001 | <0.001 |
| A | <0.001 | <0.001 |
| B | 0.012 | 0.017 |
| C | <0.001 | <0.001 |
| A*B | 0.001 | 0.002 |
| A*C | 0.035 | 0.045 |
| B*C | 0.116 | — |
| A*B*C | 0.091 | — |
| Ct Pt | <0.001 | <0.001 |

The inclusion of center points into factorial design helped to detect the curvature effect of the fitted data. Based on the aforementioned results, there was nonlinear relationship between DLE and process parameters. Consequently, response surface design was used to explore the curvature effect and to incorporate squared terms into the model to determine the optimal settings for each factor. Central composite design (CCD) is the most widely used factorial design in the response surface methodology. In addition to factorial points and center points, CCD is further augmented with a set of axial points to allow the fitting of curvature effect and to efficiently estimate the first- and second-order terms. Herein, the face-centered CCD was employed to generate the response surface model, in which axial points were at the center of the factorial surface. In CCD, the added six axial points and center points (n=4) were in another block and thus the model terms and block effects could be estimated independently, minimizing the variation of regression coefficients. Table 10 shows the change of P-values during the stepwise removement of statistically non-significant terms of the CCD model. The P-value of A*B term is 0.054, slightly greater than the significance level, indicating that the interaction between drug amount and printing speed was borderline significant. In this case, the A*B term was retained in the CCD model. Since all the lower order terms that comprise the higher order terms should also be kept in the model, factor B main effect was retained despite its P-value was marginally greater than 0.05. The final polynomial regression model for face-centered CCD was expressed by Equation 7 with R²=0.91.

$$DLE = - 79.1 + 9.22 * A + 1.35 * B + 0.596 * C - 0.0 \; 02113 {C}^{2} - 0.1519 A * B \qquad ( 7 )$$

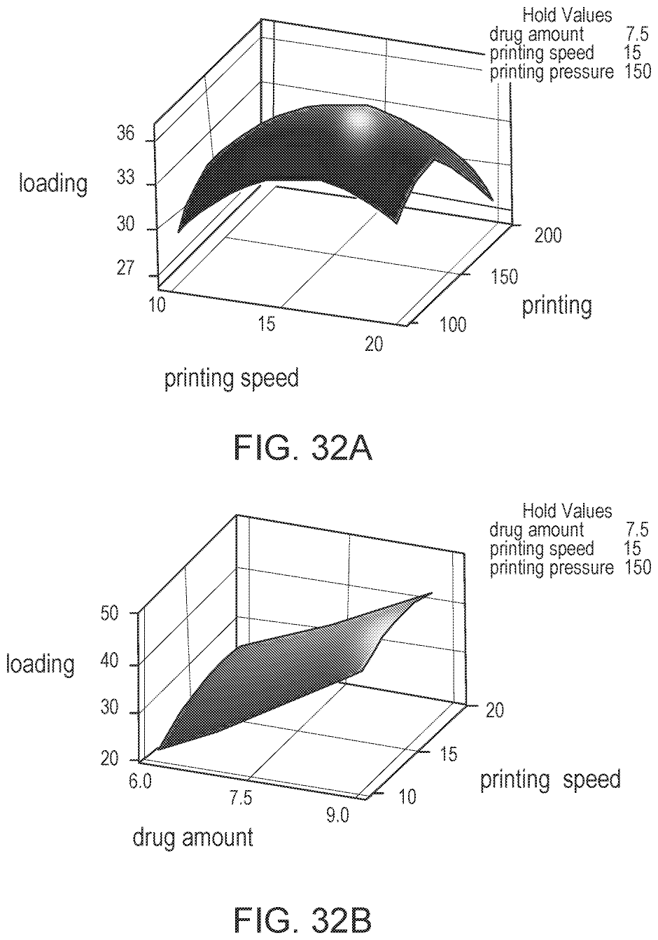

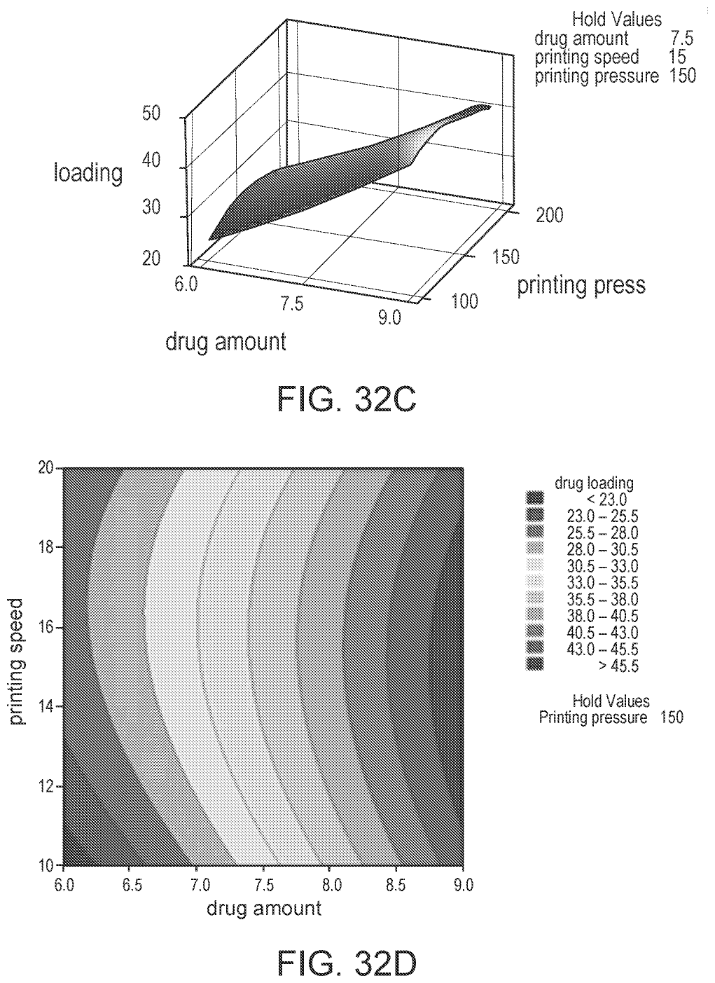

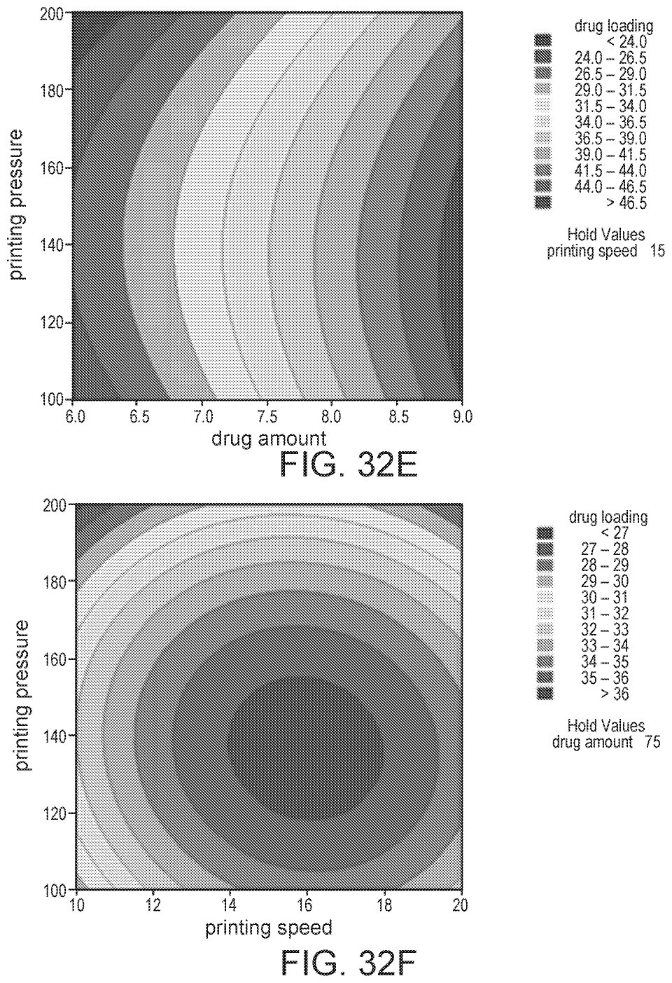

Surface plots are used to visualize the relationship between response variable and two continuous independent variables. The two factors are on the x- and y-axes, and the response variable (z) is represented by a smooth surface. As is shown in FIG. 32A, when the drug amount was held constant at 7.5 mg, there was a curvilinear relationship between DLE and printing pressure at different printing speeds. At a certain printing speed, with the increase of printing pressure, the DLE first increased to the maximum value and then decreased. The same trend was observed for the relationship between DLE and printing speed. At a certain printing pressure, an increase in the printing speed resulted in an increase of DLE to the maximum after which the DLE reduced. Additionally, FIG. 32B shows that when the printing pressure was fixed at 150 kPa, there was a linear relationship between DLE and drug amount at different printing speeds. Similarly, when the printing speed was held constant at 15 mm/s, DLE was linearly correlated with the drug amount at different printing pressures as shown in FIG. 32C. On top of that, contour plots shown in FIGS. 32D-32F were created to visualize the model equation and to display how the fitted DLE correlated with two continuous factors. In a contour plot, all points with the same response value are connected to generate the contour lines with constant responses. For instance, as shown in FIG. 32F, when the initial drug amount was fixed at 7.5 mg, with the printing speed ranging from 14.5 to 16.5 mm/s and the printing pressure ranging from 120 kPa to 150 kPa, the resulted DLE could be higher than 36 μg/mg.

TABLE 10

| | Initial P- | |
|---|---|---|
| | value (all | Final |
| | terms | P- |
| Term | included) | value |
| constant | <0.001 | <0.001 |
| Block | 0.854 | — |
| A | <0.001 | <0.001 |
| B | 0.066 | 0.065 |
| C | 0.002 | 0.002 |
| A2 | 0.588 | — |
| B2 | 0.132 | — |
| C2 | 0.075 | <0.001 |
| A*B | 0.055 | 0.054 |
| A*C | 0.225 | — |
| B*C | 0.373 | — |

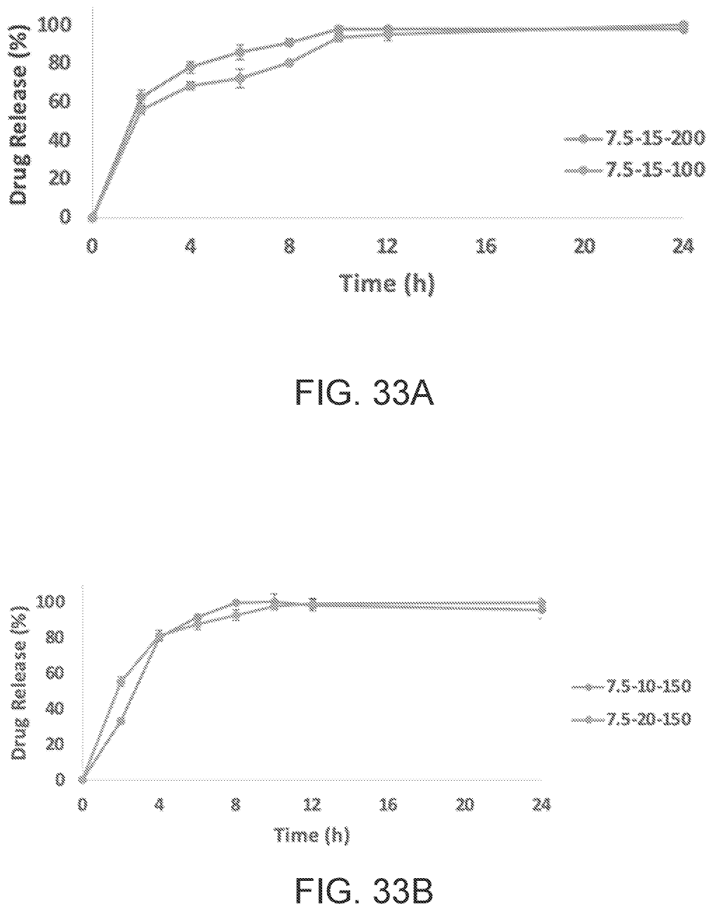

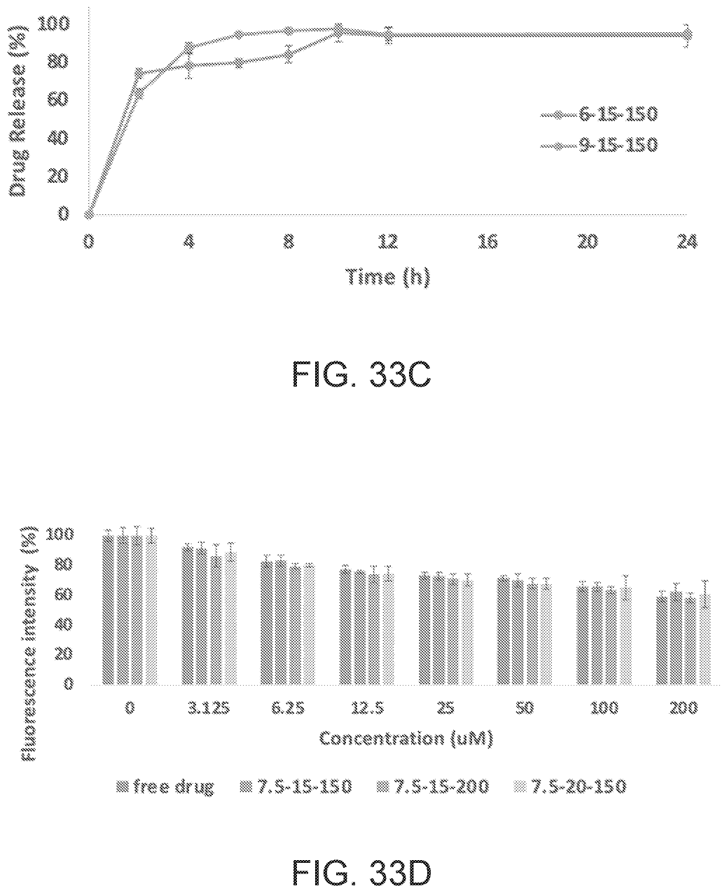

Regarding the in vitro drug release assay shown in FIGS. 33A-33C, 6-TG loaded SMART microparticles formulated under different process parameters released more than 60% payload in the first 4 h, followed by a slower release in the next 6 h, and reached ˜100% drug release at 10 h. As shown in FIG. 33A, when initial drug amount and printing speed were held constant (7.5 mg and 15 mm/s), SMART microparticles created under a higher printing speed (20 mm/s) showed a slightly faster drug release profile in the initial 2 h and a slower release in the next 8 h, compared to those created under a lower printing pressure (100 kPa). Additionally, it is shown in FIG. 33B that when initial drug amount and printing pressure were held constant (7.5 mg and 150 kPa), SMART microparticles formulated under a higher printing speed (20 mm/s) showed a faster drug release profile in the initial 4 h and a slower release in the following 8 h compared to those created under a smaller printing speed (10 mm/s). Moreover, as shown in FIG. 33C, when printing pressure and printing speed were held constant (150 kPa and 15 mm/s), SMART microparticles with a higher initial dug input (9 mg) showed a slightly slower drug release in the initial 2 h and a faster release in the next 8 h, compared to those with a smaller initial drug input (6 mg).

Furthermore, for the in vitro SARS-CoV PLpro inhibition assay, the fluorescence intensity was correlated with the PLpro activity. As shown in FIG. 33D, PLpro inhibition efficacy of 6-TG loaded SMART microparticles was comparable to free 6-TG, indicating the drug efficacy was maintained during the printing process under different printing parameters.

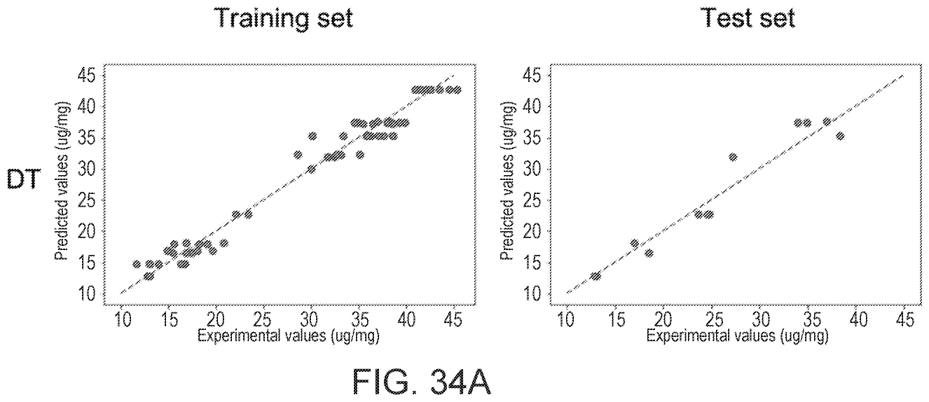

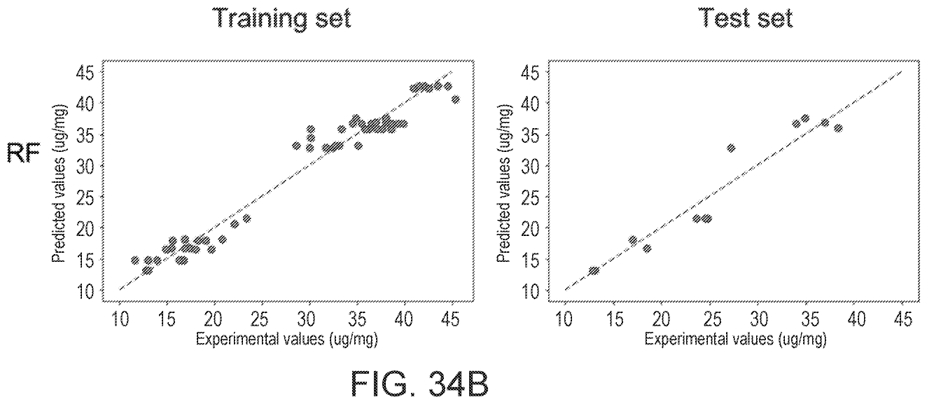

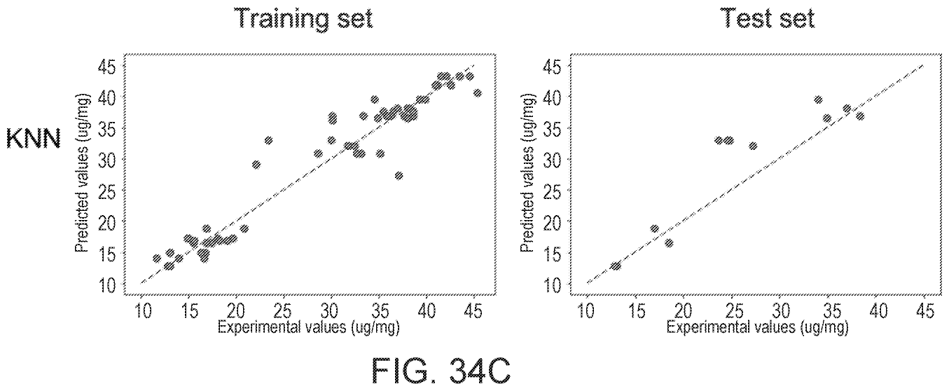

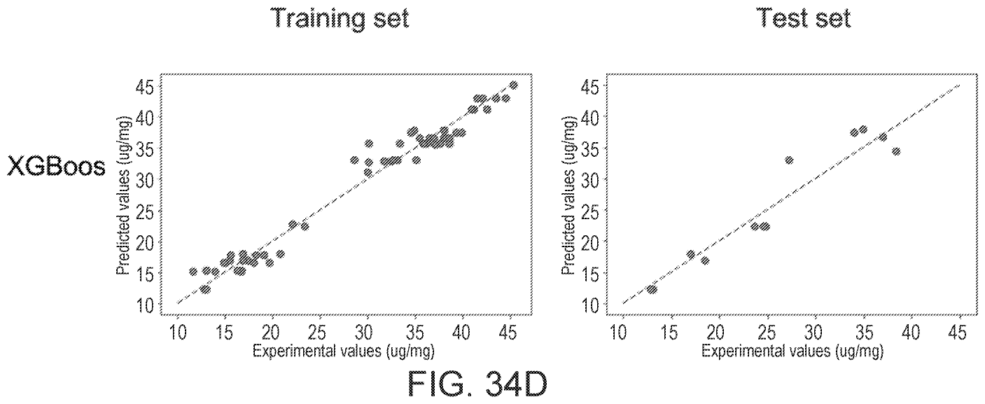

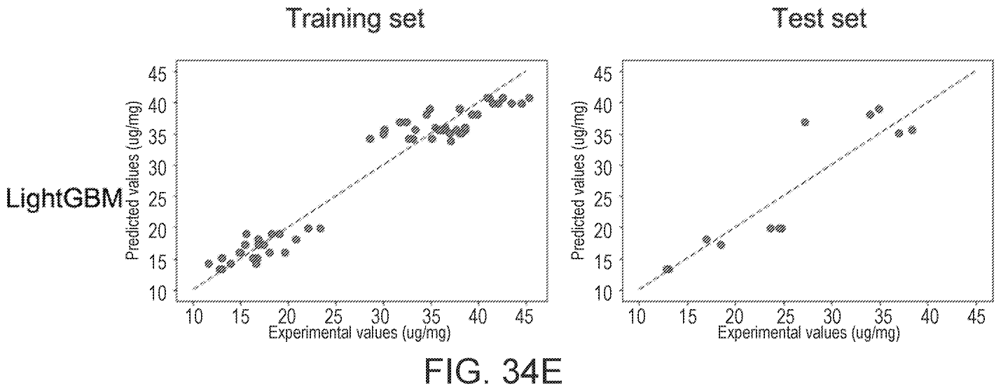

To establish the optimal predictive model for DLE, five machine learning models (DT, RF, KNN, LightGBM, XGBoost) were constructed and compared. The original dataset was split into training subset (85%) and test subset (15%). For the training subset, ten-fold cross validation method was used for model construction and tuning model hyper-parameters, while the test set was used to evaluate the model performance. Different statistical measures, including R², MAE, and RMSE, were calculated to indicate the model prediction accuracy as shown in Table 11. It was shown that DT had the smallest MAE and RMSE as well as the greatest R² among all algorithms in the test subset. These results implied that DT algorithm provided better prediction of loading efficiency than the other four algorithms. In contrast, KNN model exhibited the lowest R² and the largest MAE and RMSE values, far from the satisfied prediction for DLE. Interestingly, even though XGBoost and LightGBM are more advanced ensemble algorithms, they did not show better predictive performance than DT in the present study, which was possibly due to the small volume of dataset. This implied that conventional machine learning models might be a better fit for analyzing small datasets compared to more advanced machine learning algorithms. The scatter plots shown in FIGS. 34A-34E exhibited the predicted values calculated by different machine learning models versus the corresponding experimental values on the training and test subset.

TABLE 11

| Different | Training set | | | Test set | | |
|---|---|---|---|---|---|---|
| algorithms | R² | MAE | RMSE | R² | MAE | RMSE |
| DT | 0.969 | 1.486 | 1.877 | 0.93 | 1.869 | 2.286 |
| RF | 0.965 | 1.582 | 1.983 | 0.912 | 2.094 | 2.571 |
| KNN | 0.929 | 1.984 | 2.826 | 0.678 | 3.695 | 4.909 |
| XGBoost | 0.971 | 1.448 | 1.814 | 0.906 | 2.156 | 2.655 |
| LightGBM | 0.937 | 2.16 | 2.664 | 0.778 | 3.222 | 4.075 |

To further validate the generalization as well as the predictive accuracy of different machine learning models, new SMART microparticles with parameters that were not listed in the aforementioned DoE dataset were prepared. The process parameters and DLE were selected as the independent variables and prediction target of the validation dataset, respectively. The experimental measured DLE of SMART microparticles were compared with the predicted values calculated by the DoE regression models and the constructed machine learning models. Different statistical measures, including R², MAE, and RMSE, were calculated to signify the prediction accuracy of different DoE techniques and machine learning models on the validation dataset as shown in Table 12. It was shown that the four-factor/two-level full factorial design regression model had relatively higher R² and smaller MAE and RMSE values compared with the face-centered CCD regression model, implying that the full factorial design regression model had better predictive accuracy. The underlying reason was that CCD regression model only analyzed three continuous numerical variables without the categorical variable, nozzle size, which was also a statistically significant factor. Therefore, even though faced-centered CCD included squared terms to estimate the curvature effect of the fitted data, four-factor/two-level full factorial design model still showed superior prediction performance. Additionally, except for KNN model, the other four machine learning models (DT, RF, XGBoost, and LightGBM) all possessed higher R² and lower MAE than the two DoE regression models on the validation dataset as shown in Table 12, indicating that machine learning modeling strategies are a powerful tool to screen formulation factors and predict the microparticle performance.

Previous results proved that the fitted dataset for DoE analysis had curvature effect. Even though comprehensive DoE studies were performed to investigate the nonlinear relationship between the DLE and the four process parameters, the regression equation still could not fully analyze the complex surface curvature effect of the fitted data. Thus, tree-based machine learning models were further applied for DLE prediction due to their capacity to accommodate complex nonlinear relationships. DT, the basic tree-based machine learning algorithm, is based on a hierarchical decision scheme and consists of a root node, a set of internal nodes and a set of leaf nodes. Since the prediction target DLE is a continuous variable, a regression tree was applied to estimate DLE values. This algorithm used the binary recursive partitioning to split the data from each node into two parts to minimize the sum of the standard deviations of the separate parts. Starting from the root node, this splitting process continued until the leaf nodes were reached. RF is an ensemble learning algorithm that integrates several DT models. The output is generated based on randomly selected features and samples built on different DT models, which could avoid the data overfitting. XGBoost and LightGBM are gradient boosting algorithms, which generate the prediction models with an ensemble of weak prediction models, typically DT models. The fundamental difference between XGBoost and LightGBM is that XGBoost applies depth-wise tree growth and employs a more regularized model formalization to reduce overfitting, while LightGBM applies leaf-wise tree growth and thus has higher training speed and efficiency.

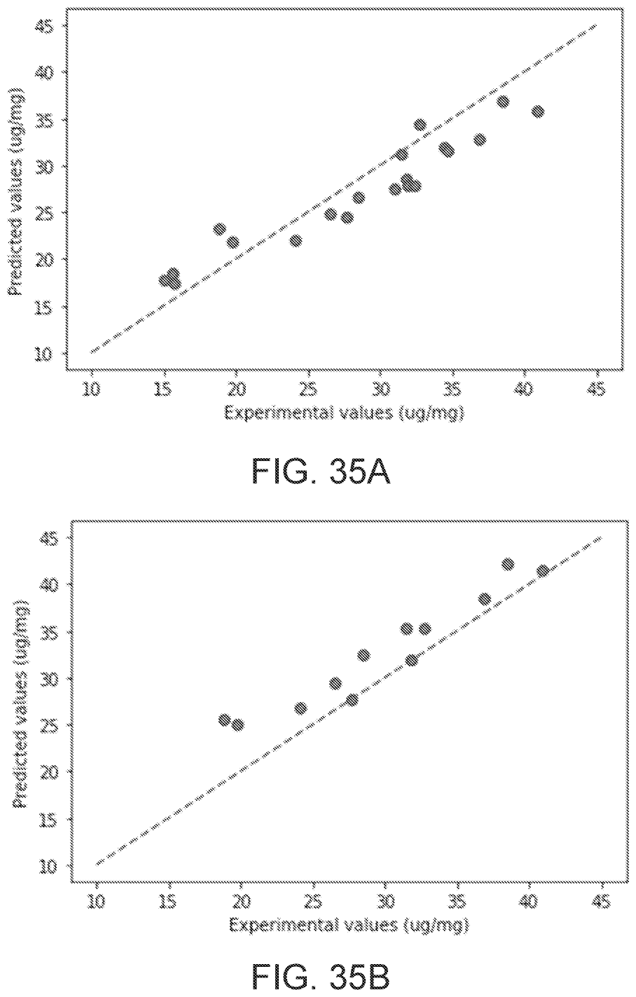

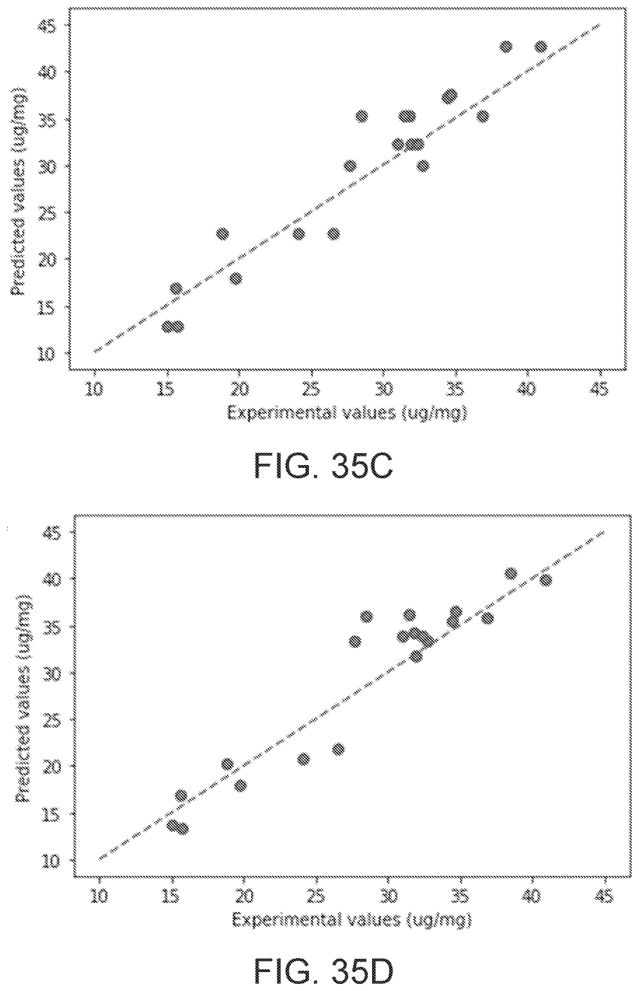

These tree-based machine learning algorithms could address both classification and regression tasks. These algorithms could be applied to accommodate linear and complex nonlinear functional relationships. Consequently, the tree-based machine learning models exhibited better curve fitting performance and higher predictive accuracy compared to DoE regression models. Moreover, in the present study, DT showed slightly better predictive performance on the validation dataset compared to the advanced ensemble algorithms, XGBoost and LightGBM. The underlying reason was that basic machine learning algorithm may be a better fit for analyzing small datasets compared to more advanced algorithms. Overall, these results indicated that machine learning modelling was an efficient approach to optimizing formulation parameters to achieve the desired microparticle properties, significantly enhancing the microparticle formulation development efficiency. The scatter plots shown in FIGS. 35A-35D exhibit the predicted values calculated by different DoE techniques and the two most predictive machine learning models versus the corresponding experimental values on the validation dataset.

TABLE 12

| | Experimental validation | | |
|---|---|---|---|
| | R² | MAE | RMSE |
| Four-factor/two-level full factorial design | 0.785 | 2.816 | 3.064 |
| Face-centered CCD | 0.776 | 2.85 | 3.445 |
| DT | 0.846 | 2.599 | 3.001 |
| RF | 0.842 | 2.436 | 3.044 |
| XGBoost | 0.836 | 2.614 | 3.1 |
| LightGBM | 0.832 | 2.41 | 3.134 |

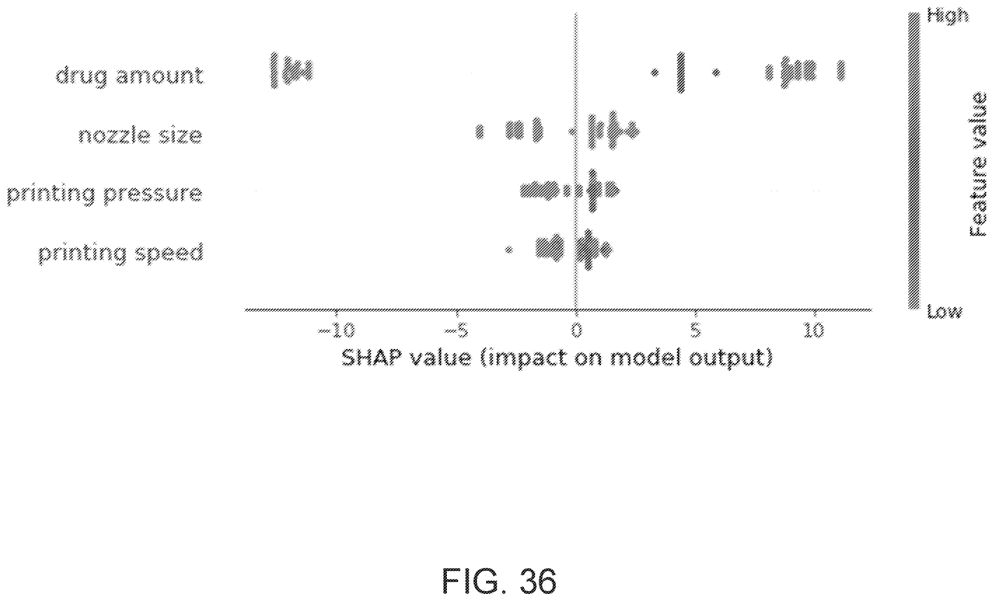

Since DT model exhibited the best prediction performance, it was selected as the final predictive model to rank the importance of formulation factors as shown in FIG. 36. The feature importance ranking could help to identify the most influential formulation factor and guide the experimental design. Among the four process parameters, initial drug amount had the most significant impact on the DLE, while nozzle size ranked the second, followed by printing pressure, and printing speed. Moreover, increasing drug amount or printing speed led to a higher DLE, whereas the increase of nozzle size or printing pressure resulted in a lower DLE, supporting the result from the main effect plot of the four-factor/two-level full factorial design as shown in FIG. 29A. Furthermore, the ranking of feature importance corresponded to the ranking of the slope in main effect plots, in which drug amount displayed the highest slope, and nozzle size came the second, followed by printing pressure and printing speed.

A 3D bioprinter was successfully used to prepare 6-TG loaded SMART microparticles by combining the emulsion solvent evaporation and extrusion-based bioprinting technique. The formulated SMART microparticles exhibited spherical structure with size distribution ˜1-3 μm in diameter. Moreover, 6-TG loaded SMART microparticles that were fabricated under different process parameters released more than 60% payload in the initial 4 h, followed by a slower release in the next 6 h, and reached ˜100% drug release at 10 h. Also, the PLpro inhibition efficacy of 6-TG was maintained during the printing process under different printing parameters. Comprehensive DoE studies were performed to optimize the SMART microparticles. In addition, the 6-TG loading efficiency of SMART microparticles was predicted by the DoE regression models and five machine learning models (DT, RF, KNN, XGBoost, and LightGBM). It was revealed that except for KNN, all the machine learning models demonstrated better predictive performance compared to the DoE regression models, which was due to their superior abilities to accommodate more complicated and nonlinear functional relationship. Moreover, DT showed the best prediction accuracy among all the learning models. According to DoE analysis and machine learning modeling, drug amount was the most influential formulation factor among the four process parameters. In summary, the use of a 3D printing concept to produce microparticle formulation is successfully presented. Furthermore, comprehensive DoE analysis and machine learning modeling were performed to predict the DLE of microparticle formulations. The predictive accuracy of different models were calculated and compared. On the other hand, the significance of process parameters was initially evaluated by DoE statistical analysis, which were further validated based on feature importance ranking by machine learning modeling. This systemic approach shows great potential in optimizing microparticle formulations with desired properties, identifying significant formulation factors, and speeding up the development of microparticles with programmable pharmaceutical attributes, representing a new paradigm for digital pharmaceutical science.

### REFERENCES

U.S. Patent Application Publication No. US 2019/0193037.
Buyukhatipoglu et al., 2009, “Bioprinted Nanoparticles for Tissue Engineering Applications,” TISSUE ENGINEERING: PART C, 16(4), 631-642. DOI: 10.1089/ten.tec.2009.0280
Yang, 2016, “Bioprinting of polymer microparticles, cells and a protein at ambient conditions,” J. 3D Pring. Med., 1(1), 81-83. DOI: 10.2217/3dp.15.2
O. Helmi, F. Elshishiny, W. Mamdouh, Targeted doxorubicin delivery and release within breast cancer environment using PEGylated chitosan nanoparticles labeled with monoclonal antibodies, Int. J. Biol. Macromol. (2021).
E. Lagreca, V. Onesto, C. Di Natale, S. La Manna, P. A. Netti, R. Vecchione, Recent advances in the formulation of PLGA microparticles for controlled drug delivery, Prog. Biomater. 9 (2020) 153-174.
O. Maksimenko, J. Malinovskaya, E. Shipulo, N. Osipova, V. Razzhivina, D. Arantseva, O. Yarovaya, U. Mostovaya, A. Khalansky, V. Fedoseeva, others, Doxorubicin-loaded PLGA nanoparticles for the chemotherapy of glioblastoma: Towards the pharmaceutical development, Int. J. Pharm. 572 (2019) 118733.
S. Cao, S. Xu, H. Wang, Y. Ling, J. Dong, R. Xia, X. Sun, Nanoparticles; oral delivery for protein and peptide drugs, AAPS PharmSciTech. 20 (2019) 1-11
W. Ho, M. Gao, F. Li, Z. Li, X.-Q. Zhang, X. Xu, Next-Generation Vaccines: Nanoparticle-Mediated DNA and mRNA Delivery, Adv. Healthc. Mater. 10 (2021) 2001812.
M. Beck-Broichsitter, E. Rytting, T. Lebhardt, X. Wang, T. Kissel, Preparation of nanoparticles by solvent displacement for drug delivery: a shift in the “ouzo region” upon drug loading, Eur. J. Pharm. Sci. 41 (2010) 244-253.
I.D. Rosca, F. Watari, M. Uo, Microparticle formation and its mechanism in single and double emulsion solvent evaporation, J. Control. Release. 99 (2004) 271-280.
M. Iqbal, N. Zafar, H. Fessi, A. Elaissari, Double emulsion solvent evaporation techniques used for drug encapsulation, Int. J. Pharm. 496 (2015) 173-190.
T. Shao, L. Bai, B. Yan, Y. Jin, Y. Cheng, Modeling the solidification of O/W-emulsion droplet in solvent evaporation technique, Chem. Eng. Res. Des. 122 (2017) 233-242.
J. Wang, Y. Zhang, N. H. Aghda, A. R. Pillai, R. Thakkar, A. Nokhodchi, M.
Maniruzzaman, Emerging 3D printing technologies for drug delivery devices: Current status and future perspective, Adv. Drug Deliv. Rev. 174 (2021) 294-316. https://doi.org/https://doi.org/10.1016/j.addr.2021.04.019.
Y. Su, B. Zhang, R. Sun, W. Liu, Q. Zhu, X. Zhang, R. Wang, C. Chen, PLGA-based biodegradable microspheres in drug delivery: recent advances in research and application, Drug Deliv. 28 (2021) 1397-1418.
H. K. Makadia, S. J. Siegel, Poly lactic-co-glycolic acid (PLGA) as biodegradable controlled drug delivery carrier, Polymers (Basel). 3 (2011) 1377-1397.
C. D. Swaim, V. Dwivedi, Y.-C. Perng, X. Zhao, L. A. Canadeo, H. H. Harastani, T. L. Darling, A. C. M. Boon, D. J. Lenschow, V. Kulkarni, others, 6-Thioguanine blocks SARS-CoV-2 replication by inhibition of PLpro, Iscience. 24 (2021) 103213.
V. Czitrom, One-factor-at-a-time versus designed experiments, Am. Stat. 53 (1999) 126-131.
L. Eriksson, E. Johansson, N. Kettaneh-Wold, C. Wikstrom, S. Wold, Design of experiments, Princ. Appl. Learn Ways AB, Stock. (2000).
V. Nekkanti, A. Marwah, R. Pillai, Media milling process optimization for manufacture of drug nanoparticles using design of experiments (DOE), Drug Dev. Ind. Pharm. 41 (2015) 124-130.
S. Beg, S. Swain, M. Rahman, M. S. Hasnain, S. S. Imam, Application of design of experiments (DoE) in pharmaceutical product and process optimization, in: Pharm. Qual. by Des., Elsevier, 2019: pp. 43-64.
G. Gujral, D. Kapoor, M. Jaimini, An updated review on design of experiment (DOE) in pharmaceuticals, J. Drug Deliv. Ther. 8 (2018) 147-152.
S. Bhattacharya, Central composite design for response surface methodology and its application in pharmacy, (2021).
G. Singh, R. S. Pai, V. K. Devi, Response surface methodology and process optimization of sustained release pellets using Taguchi orthogonal array design and central composite design, J. Adv. Pharm. Technol. & Res. 3 (2012) 30.
J. Molpeceres, M. Guzman, M. R. Aberturas, M. Chacon, L. Berges, Application of central composite designs to the preparation of polycaprolactone nanoparticles by solvent displacement, J. Pharm. Sci. 85 (1996) 206-213.
S. A. Damiati, Digital Pharmaceutical Sciences, AAPS PharmSciTech. 21 (2020) 1-12.
Y. He, Z. Ye, X. Liu, Z. Wei, F. Qiu, H.-F. Li, Y. Zheng, D. Ouyang, Can machine learning predict drug nanocrystals?, J. Control. Release. 322 (2020) 274-285.
R. A. Hashad, R. A. H. Ishak, S. Fahmy, S. Mansour, A. S. Geneidi, Chitosan-tripolyphosphate nanoparticles: Optimization of formulation parameters for improving process yield at a novel pH using artificial neural networks, Int. J. Biol. Macromol. 86 (2016) 50-58.
Y. Li, M. R. Abbaspour, P. V Grootendorst, A. M. Rauth, X. Y. Wu, Optimization of controlled release nanoparticle formulation of verapamil hydrochloride using artificial neural networks with genetic algorithm and response surface methodology, Eur. J. Pharm. Biopharm. 94 (2015) 170-179.
J. D. Brown, The coefficient of determination, (2009).
C. J. Willmott, K. Matsuura, Advantages of the mean absolute error (MAE) over the root mean square error (RMSE) in assessing average model performance, Clim. Res. 30 (2005) 79-82.
T. Chai, R. R. Draxler, Root mean square error (RMSE) or mean absolute error (MAE), Geosci. Model Dev. Discuss. 7 (2014) 1525-1534.
S. A. M. Martins, D. M. F. Prazeres, L. P. Fonseca, G. A. Monteiro, Application of central composite design for DNA hybridization onto magnetic microparticles, Anal. Biochem. 391 (2009) 17-23.
D. C. Montgomery, Design and analysis of experiments, John wiley & sons, 2017.
W. C. t Hamilton, S. C. Abrahams, Normal probability plot analysis of small samples, Acta Crystallogr. Sect. A Cryst. Physics, Diffraction, Theor. Gen. Crystallogr. 28 (1972) 215-218.
G. C. J. Fernandez, Residual analysis and data transformations: important tools in statistical analysis, HortScience. 27 (1992) 297-300.
J. Antony, Design of experiments for engineers and scientists, Elsevier, 2014.
R. Mukerjee, C.-F. Wu, A modern theory of factorial design, Springer, 2006.
R. Splechtna, M. Elshehaly, D. Gracanin, M. Duras, K. Buhler, K. Matkovid, Interactive interaction plot, Vis. Comput. 31 (2015) 1055-1065.
S. N. Politis, P. Colombo, G. Colombo, D. M. Rekkas, Design of experiments (DoE) in pharmaceutical development, Drug Dev. Ind. Pharm. 43 (2017) 889-901.
M. Balachandran, S. Devanathan, R. Muraleekrishnan, S. S. Bhagawan, Optimizing properties of nanoclay—nitrile rubber (NBR) composites using face centred central composite design, Mater. & Des. 35 (2012) 854-862.
E. D. Madyatmadja, A. Rianto, J. F. Andry, H. Tannady, A. Chakir, Analysis of Big Data in Healthcare Using Decision Tree Algorithm, in: 2021 1st Int. Conf Comput. Sci. Artif. Intell., 2021: pp. 313-317.
J. Ronowicz, M. Thommes, P. Kleinebudde, J. Krysinski, A data mining approach to optimize pellets manufacturing process based on a decision tree algorithm, Eur. J. Pharm. Sci. 73 (2015) 44-48.
M. Xu, P. Watanachaturapom, P. K. Varshney, M. K. Arora, Decision tree regression for soft classification of remote sensing data, Remote Sens. Environ. 97 (2005) 322-336.
R. Han, H. Xiong, Z. Ye, Y. Yang, T. Huang, Q. Jing, J. Lu, H. Pan, F. Ren, D. Ouyang, Predicting physical stability of solid dispersions by machine learning techniques, J. Control. Release. 311 (2019) 16-25.
Y. Yang, Z. Ye, Y. Su, Q. Zhao, X. Li, D. Ouyang, Deep learning for in vitro prediction of pharmaceutical formulations, Acta Pharm. Sin. B. 9 (2019) 177-185.
T. Hastie, R. Tibshirani, J. Friedman, Boosting and additive trees, in: Elem. Stat. Learn., Springer, 2009: pp. 337-387.
J. Dong, H. Gao, D. Ouyang, PharmSD: A novel AI-based computational platform for solid dispersion formulation design, Int. J. Pharm. 604 (2021) 120705.
E. Al Daoud, Comparison between XGBoost, LightGBM and CatBoost using a home credit dataset, Int. J. Comput. Inf. Eng. 13 (2019) 6-10.

### STATEMENTS REGARDING INCORPORATION BY REFERENCE AND VARIATIONS

All references throughout this application, for example, patent documents including issued or granted patents or equivalents; patent application publications; and non-patent literature documents or other source material; are hereby incorporated by reference herein in their entireties, as though individually incorporated by reference.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art.

When a group of substituents is disclosed herein, it is understood that all individual members of those groups and all subgroups and classes that can be formed using the substituents are disclosed separately. When a Markush group or other grouping is used herein, all individual members of the group and all combinations and sub-combinations possible of the group are intended to be individually included in the disclosure. As used herein, “and/or” means that one, all, or any combination of items in a list separated by “and/or” are included in the list; for example, “1, 2, and/or 3” is equivalent to “1, 2, 3, 1 and 2, 1 and 3, 2 and 3, or 1, 2 and 3”.

Every formulation or combination of components described or exemplified can be used to practice the invention unless otherwise stated. Specific names of materials are intended to be exemplary, as it is known that one of ordinary skill in the art can name the same material differently. It will be appreciated that methods, device elements, starting materials, and synthetic methods other than those specifically exemplified can be employed in the practice of the invention without resorting to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges, and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element, elements, limitation, or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art and that such modifications and variations are considered to be within the scope of this invention as defined by the appended claims.

1. A method comprising:
preparing an emulsion comprising water, a polymer or a non-polymeric excipient, a solvent, and an active pharmaceutical ingredient;
printing the emulsion using an extrusion-based printing method to generate a plurality of droplets including particles having diameters of from 10 nm to 1100 μm and comprising the polymer or the non-polymeric excipient and the active pharmaceutical ingredient; and
collecting the plurality of droplets.

2. The method of claim 1, wherein the extrusion-based printing method subjects the emulsion to shear forces that separate the emulsion into the plurality of droplets including particles.

3. The method of claim 1, further comprising subjecting the droplets to evaporation conditions to evaporate from the droplets and leave the particles.

4. The method of claim 3, further comprising washing the plurality of particles.

5. (canceled)

6. The method of claim 1, wherein the extrusion-based printing method comprises one or more of emulsion-evaporation/diffusion, nanoprecipitation, desolvation, gelation, or spray-based atomization.

7. The method of claim 1, wherein preparing the emulsion comprises preparing a primary emulsion comprising a water-in-oil emulsion or an oil-in-water emulsion, and preparing a secondary emulsion comprising a water-in-oil-in-water emulsion.

8. The method of claim 1, wherein the emulsion comprises or further comprises one or more of a cosolvent, a surfactant, a preservative, live cells, cellular components, an additional active ingredient, a salt, a preservative, a protein, a peptide, an amino acid, or a nucleic acid component.

9. The method of claim 1,
wherein the active ingredient comprises a protein, an antibody, a nucleic acid, messenger ribonucleic acid (mRNA) molecules, a lipid nanoparticle, clustered regularly interspaced short palindromic repeats (CRISPR)-associated protein 9 (Cas9), transcription activator-like effector nucleases (TALENs), zinc-finger nucleases (ZFNs), homing endonucleases or meganucleases, a growth factor, a plasmid, a hydrophilic pharmaceutical, a lipophilic pharmaceutical, a viral particle, a virus-like particle, a live yeast cell, a live recombinant yeast cell, a live fungus, a live bacterial cell, a live recombinant bacterial cell, a live insect cell, a live mammalian cell, or a live mesenchymal stem cell; or
wherein the polymer is a biodegradable polymer selected from the group consisting of poly(lactide-co-glycolide), polylactide (PLA), polyglycolide (PGA), polycaprolactone (PCL), pluronic F127, sodium alginate, hyaluronic acid, chitosan, cyclodextrin, dextran, agarose, gelatin, albumin, collagen, lipids, a polyethylene glycol (PEG) derivative, a pharmaceutical grade polymer, poly(hydroxy butyrate), poly(β-malic acid), or poly(L-lysine); or
wherein the non-polymeric excipient is a hydrophilic substance, a hydrophobic substance, a non-reducing sugar, trehalose, sucrose, a polyol, mannitol, sorbitol, xylitol, an amino acid, leucine, or L-arginine.

10. (canceled)

11. (canceled)

12. The method of claim 1, wherein collecting the plurality of droplets comprises receiving the plurality of droplets on a surface having a temperature of from about −200° C. to about room temperature.

13. The method of claim 1,
wherein the extrusion-based printing method subjects the emulsion to a pressure of from 10 kPa to 700 kPa; or
wherein the extrusion-based printing method uses a nozzle having a diameter of from 1 μm to 1000 μm; or
wherein an extrusion pressure of the extrusion-based printing method greater than or about 200 kPa; or
wherein a temperature of the emulsion during the printing is from about 4° C. to about 50° C.; or
wherein printing the emulsion comprises receiving the particles on a surface, wherein the surface has a temperature of about room temperature or less than or about −78° C.; or
wherein a weight ratio of the active pharmaceutical ingredient to the polymer or the non-polymeric excipient in the emulsion is from 1:8 to 1:15.

14.-18. (canceled)

19. The method of claim 1, further comprising lyophilizing the plurality of droplets or the particles.

20. A system comprising:
an emulsion supply container for preparing or storing an emulsion comprising water, a polymer or a non-polymeric excipient, a solvent, and an active pharmaceutical ingredient;
one or more extrusion-based printing nozzles in fluid communication with the emulsion supply container for generating a plurality of droplets of the emulsion including particles having diameters of from 10 nm to 1100 μm; and
a collection surface for receiving the plurality of droplets of the emulsion from the one or more extrusion-based printing nozzles.

21. The system of claim 20, wherein the collection surface is cooled to a temperature of from about −200° C. to about −75° C., or wherein the system further comprises a cooling or refrigeration system coupled to the collection surface for cooling the collection surface to a temperature of from about −200° C. to about −75° C.

22. (canceled)

23. The system of claim 20, further comprising one or more of:
one or more temperature sensors or temperature controllers for monitoring or controlling a temperature of the collection surface; or
a translation stage for generating a relative translation between the one or more extrusion-based printing nozzles and the collection surface; or
one or more mixing vessels in fluid communication with the emulsion supply container for preparing and providing the emulsion to the emulsion supply container; or
one or more pressure sensors or pressure controllers for monitoring or controlling an extrusion pressure associated with the one or more extrusion-based printing nozzles; or one or more actuators for monitoring or controlling an extrusion speed associated with the one or more extrusion-based printing nozzles; or
a housing for maintaining at least the one or more extrusion-based printing nozzles and the collection surface in a sterile environment; or
sterilization equipment positioned to sterilize one or more of the an emulsion supply container, the one or more extrusion-based printing nozzles, or the collection surface.

24. The system of claim 20, wherein the collection surface is a moving or translating collection surface, or wherein the collection surface comprises a sterile vile.

25.-31. (canceled)

32. A composition comprising:
particles comprising a polymer or a non-polymeric excipient, the particles having diameters of from 10 nm to 1000 μm; and
one or more live cells, wherein:
the particles are attached to surfaces of the one or more live cells, or
the one or more live cells are at least partially encapsulated into the particles.

33. The composition of claim 32, wherein the particles further comprise an active ingredient embedded within or adsorbed to the particles.

34. The composition of claim 33, wherein the active ingredient comprises a protein, an antibody, a nucleic acid, messenger ribonucleic acid (mRNA) molecules, a lipid nanoparticle, clustered regularly interspaced short palindromic repeats (CRISPR)-associated protein 9 (Cas9), transcription activator-like effector nucleases (TALENs), zinc-finger nucleases (ZFNs), homing endonucleases or meganucleases, a growth factor, a plasmid, a hydrophilic pharmaceutical, a lipophilic pharmaceutical, a viral particle, a virus-like particle, a live yeast cell, a live recombinant yeast cell, a live fungus, a live bacterial cell, a live recombinant bacterial cell, a live insect cell, a live mammalian cell, or a live mesenchymal stem cell.

35. The composition of claim 32,
wherein the polymer is a biodegradable polymer selected from the group consisting of poly(lactide-co-glycolide), polylactide (PLA), polyglycolide (PGA), polycaprolactone (PCL), pluronic F127, sodium alginate, hyaluronic acid, chitosan, cyclodextrin, dextran, agarose, gelatin, albumin, collagen, lipids, a polyethylene glycol (PEG) derivative, a pharmaceutical grade polymer, poly(hydroxy butyrate), poly(β-malic acid), or poly(L-lysine); or
wherein the non-polymeric excipient is a hydrophilic substance, a hydrophobic substance, a non-reducing sugar, trehalose, sucrose, a polyol, mannitol, sorbitol, xylitol, an amino acid, leucine, or L-arginine; or
wherein one or more live cells comprise live yeast cells, live recombinant yeast cells, live fungal cells, live bacterial cells, live recombinant bacterial cells, live insect cells, live mammalian cells, or live mesenchymal stem cells.

36. (canceled)

37. (canceled)

38. The composition of claim 32, wherein the particles are in a lyophilized condition.
